SEQUENCE LISTING (12 sequences)

SEQ ID NO: 1 - RNA, 15 nt, synthetic construct
  source 1..15: mol_type = other RNA
  modified_base 1: mod_base = OTHER; note = 2 prime fluorouridine
  modified_base 2: mod_base = OTHER; note = 2 prime-O-methyl guanosine
  modified_base 3: mod_base = OTHER; note = 2 prime fluorouridine
  modified_base 4: mod_base = OTHER; note = 2 prime-O-methyl guanosine
  modified_base 5: mod_base = OTHER; note = 2 prime fluoroguanosine
  modified_base 6: mod_base = OTHER; note = 2 prime-O-methyl uridine
  modified_base 7: mod_base = OTHER; note = 2 prime fluorouridine
  modified_base 8: mod_base = OTHER; note = 2 prime-O-methyl cytidine
  modified_base 9: mod_base = OTHER; note = 2 prime fluorouridine
  modified_base 10: mod_base = OTHER; note = 2 prime-O-methyl adenosine
  modified_base 11: mod_base = OTHER; note = 2 prime fluorouridine
  modified_base 12: mod_base = OTHER; note = 2 prime-O-methyl uridine
  modified_base 13: mod_base = OTHER; note = 2 prime fluoroadenosine
  modified_base 14: mod_base = OTHER; note = 2 prime-O-methyl uridine
  modified_base 15: mod_base = OTHER; note = 2 prime fluoroadenosine
  misc_feature 13^14: note = phosphorothioate linkage
  misc_feature 14^15: note = phosphorothioate linkage
tgtggttctattata

SEQ ID NO: 2 - RNA, 20 nt, synthetic construct
  source 1..20: mol_type = other RNA
  modified_base 1: mod_base = OTHER; note = 2 prime-O-methyl uridine
  misc_feature 1..3: note = phosphorothioate linkage
  modified_base 3: mod_base = OTHER; note = 2 prime-O-methyl uridine
  modified_base 4..5: mod_base = OTHER; note = 2 prime-O-methyl adenosine
  modified_base 6: mod_base = OTHER; note = 2 prime fluorouridine
  modified_base 7: mod_base = OTHER; note = 2 prime-O-methyl adenosine
  modified_base 8: mod_base = OTHER; note = 2 prime-O-methyl guanosine
  modified_base 9: mod_base = OTHER; note = 2 prime-O-methyl adenosine
  modified_base 10: mod_base = OTHER; note = 2 prime fluoroadenosine
  modified_base 11..12: mod_base = OTHER; note = 2 prime-O-methyl cytidine
  modified_base 13: mod_base = OTHER; note = 2 prime-O-methyl adenosine
  modified_base 14: mod_base = OTHER; note = 2 prime fluorocytidine
  modified_base 15: mod_base = OTHER; note = 2 prime-O-methyl adenosine
  modified_base 16: mod_base = OTHER; note = 2 prime fluoroguanosine
  modified_base 17..18: mod_base = OTHER; note = 2 prime-O-methyl guanosine
  modified_base 19..20: mod_base = OTHER; note = 2 prime-O-methyl adenosine
  misc_feature 13..20: note = phosphorothioate linkage
tataatagaaccacagggaa

SEQ ID NO: 3 - RNA, 20 nt, synthetic construct
  source 1..20: mol_type = other RNA
  modified_base 1: mod_base = OTHER; note = 2 prime-O-methyl uridine
  misc_feature 1..3: note = phosphorothioate linkage
  modified_base 2: mod_base = OTHER; note = 2 prime-O-methyl adenosine
  modified_base 3: mod_base = OTHER; note = 2 prime-O-methyl uridine
  modified_base 4: mod_base = OTHER; note = 2 prime fluoroadenosine
  modified_base 5: mod_base = OTHER; note = 2 prime-O-methyl adenosine
  modified_base 6: mod_base = OTHER; note = 2 prime fluorouridine
  modified_base 7: mod_base = OTHER; note = 2 prime-O-methyl adenosine
  modified_base 8: mod_base = OTHER; note = 2 prime-O-methyl guanosine
  modified_base 9: mod_base = OTHER; note = 2 prime-O-methyl adenosine
  modified_base 10: mod_base = OTHER; note = 2 prime fluoroadenosine
  modified_base 11..12: mod_base = OTHER; note = 2 prime-O-methyl cytidine
  modified_base 13: mod_base = OTHER; note = 2 prime-O-methyl adenosine
  modified_base 14: mod_base = OTHER; note = 2 prime-O-methyl cytidine
  modified_base 15: mod_base = OTHER; note = 2 prime-O-methyl adenosine
  modified_base 16: mod_base = OTHER; note = 2 prime fluoroguanosine
  modified_base 17..18: mod_base = OTHER; note = 2 prime-O-methyl guanosine
  modified_base 19..20: mod_base = OTHER; note = 2 prime-O-methyl adenosine
  misc_feature 13..20: note = phosphorothioate linkage
tataatagaaccacagggaa

SEQ ID NO: 4 - RNA, 20 nt, synthetic construct
  source 1..20: mol_type = other RNA
  modified_base 1: mod_base = OTHER; note = 2 prime-O-methyl uridine
  misc_feature 1..3: note = phosphorothioate linkage
  modified_base 2: mod_base = OTHER; note = 2 prime fluoroadenosine
  modified_base 3: mod_base = OTHER; note = 2 prime-O-methyl uridine
  modified_base 4..5: mod_base = OTHER; note = 2 prime-O-methyl adenosine
  modified_base 6: mod_base = OTHER; note = 2 prime fluorouridine
  modified_base 7: mod_base = OTHER; note = 2 prime-O-methyl adenosine
  modified_base 8: mod_base = OTHER; note = 2 prime-O-methyl guanosine
  modified_base 9: mod_base = OTHER; note = 2 prime-O-methyl adenosine
  modified_base 11..12: mod_base = OTHER; note = 2 prime-O-methyl cytidine
  modified_base 13: mod_base = OTHER; note = 2 prime-O-methyl adenosine
  modified_base 14: mod_base = OTHER; note = 2 prime fluorocytidine
  modified_base 15: mod_base = OTHER; note = 2 prime-O-methyl adenosine
  modified_base 16: mod_base = OTHER; note = 2 prime fluoroguanosine
  modified_base 17..18: mod_base = OTHER; note = 2 prime-O-methyl guanosine
  modified_base 19..20: mod_base = OTHER; note = 2 prime-O-methyl adenosine
  misc_feature 13..20: note = phosphorothioate linkage
tataatagaaccacagggaa

SEQ ID NO: 5 - RNA, 20 nt, synthetic construct
  source 1..20: mol_type = other RNA
  modified_base 1: mod_base = OTHER; note = 2 prime-O-methyl uridine
  misc_feature 1..3: note = phosphorothioate linkage
  modified_base 2: mod_base = OTHER; note = 2 prime fluoroadenosine
  modified_base 3: mod_base = OTHER; note = 2 prime-O-methyl uridine
  modified_base 4: mod_base = OTHER; note = 2 prime fluoroadenosine
  modified_base 5: mod_base = OTHER; note = 2 prime-O-methyl adenosine
  modified_base 6: mod_base = OTHER; note = 2 prime fluorouridine
  modified_base 7: mod_base = OTHER; note = 2 prime-O-methyl adenosine
  modified_base 9: mod_base = OTHER; note = 2 prime-O-methyl adenosine
  modified_base 10: mod_base = OTHER; note = 2 prime fluoroadenosine
  modified_base 11: mod_base = OTHER; note = 2 prime-O-methyl cytidine
  modified_base 12: mod_base = OTHER; note = 2 prime fluorocytidine
  modified_base 14: mod_base = OTHER; note = 2 prime-O-methyl cytidine
  modified_base 15: mod_base = OTHER; note = 2 prime-O-methyl adenosine
  modified_base 16: mod_base = OTHER; note = 2 prime fluoroguanosine
  modified_base 17..18: mod_base = OTHER; note = 2 prime-O-methyl guanosine
  modified_base 19..20: mod_base = OTHER; note = 2 prime-O-methyl adenosine
  misc_feature 13..20: note = phosphorothioate linkage
tataatagaaccacagggaa

SEQ ID NO: 6 - RNA, 20 nt, synthetic construct
  source 1..20: mol_type = other RNA
  modified_base 1: mod_base = OTHER; note = 2 prime-O-methyl uridine
  misc_feature 1..3: note = phosphorothioate linkage
  modified_base 2: mod_base = OTHER; note = 2 prime fluoroadenosine
  modified_base 3: mod_base = OTHER; note = 2 prime-O-methyl uridine
  modified_base 4: mod_base = OTHER; note = 2 prime fluoroadenosine
  modified_base 5: mod_base = OTHER; note = 2 prime-O-methyl adenosine
  modified_base 6: mod_base = OTHER; note = 2 prime fluorouridine
  modified_base 7: mod_base = OTHER; note = 2 prime-O-methyl adenosine
  modified_base 9: mod_base = OTHER; note = 2 prime-O-methyl adenosine
  modified_base 10: mod_base = OTHER; note = 2 prime fluoroadenosine
  modified_base 11: mod_base = OTHER; note = 2 prime-O-methyl cytidine
  modified_base 12: mod_base = OTHER; note = 2 prime fluorocytidine
  modified_base 14: mod_base = OTHER; note = 2 prime fluorocytidine
  modified_base 15: mod_base = OTHER; note = 2 prime-O-methyl adenosine
  modified_base 16: mod_base = OTHER; note = 2 prime fluoroguanosine
  modified_base 17..18: mod_base = OTHER; note = 2 prime-O-methyl guanosine
  modified_base 19..20: mod_base = OTHER; note = 2 prime-O-methyl adenosine
  misc_feature 13..20: note = phosphorothioate linkage
tataatagaaccacagggaa

SEQ ID NO: 7 - RNA, 20 nt, synthetic construct
  source 1..20: mol_type = other RNA
  modified_base 1: mod_base = OTHER; note = 2 prime-O-methyl uridine
  misc_feature 1..3: note = phosphorothioate linkage
  modified_base 3: mod_base = OTHER; note = 2 prime-O-methyl uridine
  modified_base 4: mod_base = OTHER; note = 2 prime-O-methyl adenosine
  modified_base 5: mod_base = OTHER; note = 2 prime-O-methyl adenosine
  modified_base 6: mod_base = OTHER; note = 2 prime fluorouridine
  modified_base 7: mod_base = OTHER; note = 2 prime-O-methyl adenosine
  modified_base 8: mod_base = OTHER; note = 2 prime-O-methyl guanosine
  modified_base 9: mod_base = OTHER; note = 2 prime-O-methyl adenosine
  modified_base 11: mod_base = OTHER; note = 2 prime fluorocytidine
  modified_base 12: mod_base = OTHER; note = 2 prime-O-methyl cytidine
  modified_base 13: mod_base = OTHER; note = 2 prime-O-methyl adenosine
  modified_base 14: mod_base = OTHER; note = 2 prime fluorocytidine
  modified_base 15: mod_base = OTHER; note = 2 prime-O-methyl adenosine
  modified_base 16: mod_base = OTHER; note = 2 prime fluoroguanosine
  modified_base 17..18: mod_base = OTHER; note = 2 prime-O-methyl guanosine
  modified_base 19..20: mod_base = OTHER; note = 2 prime-O-methyl adenosine
  misc_feature 13..20: note = phosphorothioate linkage
tataatagaaccacagggaa

SEQ ID NO: 8 - RNA, 20 nt, synthetic construct
  source 1..20: mol_type = other RNA
  modified_base 1: mod_base = OTHER; note = 2 prime-O-methyl uridine
  modified_base 2: mod_base = OTHER; note = 2 prime fluoroadenosine
  misc_feature 1..3: note = phosphorothioate linkage
  modified_base 3: mod_base = OTHER; note = 2 prime-O-methyl uridine
  modified_base 4: mod_base = OTHER; note = 2 prime fluoroadenosine
  modified_base 5: mod_base = OTHER; note = 2 prime-O-methyl adenosine
  modified_base 6: mod_base = OTHER; note = 2 prime fluorouridine
  modified_base 7: mod_base = OTHER; note = 2 prime-O-methyl adenosine
  modified_base 8: mod_base = OTHER; note = 2 prime fluoroguanosine
  modified_base 9: mod_base = OTHER; note = 2 prime-O-methyl adenosine
  modified_base 10: mod_base = OTHER; note = 2 prime fluoroadenosine
  modified_base 11: mod_base = OTHER; note = 2 prime-O-methyl cytidine
  modified_base 12: mod_base = OTHER; note = 2 prime-O-methyl cytidine
  modified_base 13: mod_base = OTHER; note = 2 prime-O-methyl adenosine
  modified_base 14: mod_base = OTHER; note = 2 prime fluorocytidine
  modified_base 15: mod_base = OTHER; note = 2 prime-O-methyl adenosine
  modified_base 16..18: mod_base = OTHER; note = 2 prime-O-methyl guanosine
  modified_base 19..20: mod_base = OTHER; note = 2 prime-O-methyl adenosine
  misc_feature 13..20: note = phosphorothioate linkage
tataatagaaccacagggaa

SEQ ID NO: 9 - RNA, 15 nt, synthetic construct
  source 1..15: mol_type = other RNA
  modified_base 1: mod_base = OTHER; note = 2 prime-O-methyl uridine
  modified_base 2: mod_base = OTHER; note = 2 prime-O-methyl guanosine
  modified_base 3: mod_base = OTHER; note = 2 prime fluorouridine
  misc_feature 1..3: note = phosphorothioate linkage
  modified_base 4..5: mod_base = OTHER; note = 2 prime fluoroguanosine
  modified_base 6: mod_base = OTHER; note = 2 prime-O-methyl uridine
  modified_base 7: mod_base = OTHER; note = 2 prime fluorouridine
  modified_base 8: mod_base = OTHER; note = 2 prime-O-methyl cytidine
  modified_base 9: mod_base = OTHER; note = 2 prime-O-methyl uridine
  modified_base 10: mod_base = OTHER; note = 2 prime-O-methyl adenosine
  modified_base 11: mod_base = OTHER; note = 2 prime fluorouridine
  modified_base 12: mod_base = OTHER; note = 2 prime-O-methyl uridine
  modified_base 13: mod_base = OTHER; note = 2 prime-O-methyl adenosine
  modified_base 14: mod_base = OTHER; note = 2 prime-O-methyl uridine
  modified_base 15: mod_base = OTHER; note = 2 prime-O-methyl adenosine
  misc_feature 13^14: note = phosphorothioate linkage
  misc_feature 14^15: note = phosphorothioate linkage
tgtggttctattata

SEQ ID NO: 10 - RNA, 20 nt, synthetic construct
  source 1..20: mol_type = other RNA
  modified_base 1: mod_base = OTHER; note = 2 prime-O-methyl uridine
  modified_base 2: mod_base = OTHER; note = 2 prime fluoroadenosine
  modified_base 3: mod_base = OTHER; note = 2 prime fluorouridine
  modified_base 4: mod_base = OTHER; note = 2 prime-O-methyl adenosine
  modified_base 5: mod_base = OTHER; note = 2 prime-O-methyl adenosine
  modified_base 6: mod_base = OTHER; note = 2 prime fluorouridine
  modified_base 7: mod_base = OTHER; note = 2 prime-O-methyl adenosine
  modified_base 8: mod_base = OTHER; note = 2 prime-O-methyl guanosine
  modified_base 9: mod_base = OTHER; note = 2 prime-O-methyl adenosine
  modified_base 10: mod_base = OTHER; note = 2 prime fluoroadenosine
  modified_base 11..12: mod_base = OTHER; note = 2 prime fluorocytidine
  modified_base 13: mod_base = OTHER; note = 2 prime-O-methyl adenosine
  modified_base 14: mod_base = OTHER; note = 2 prime fluorocytidine
  modified_base 15: mod_base = OTHER; note = 2 prime-O-methyl adenosine
  modified_base 16..18: mod_base = OTHER; note = 2 prime-O-methyl guanosine
  modified_base 19..20: mod_base = OTHER; note = 2 prime-O-methyl adenosine
  misc_feature 13..20: note = phosphorothioate linkage
tataatagaaccacagggaa

SEQ ID NO: 11 - RNA, 15 nt, synthetic construct
  source 1..15: mol_type = other RNA
  modified_base 1: mod_base = OTHER; note = 2 prime-O-methyl uridine
  modified_base 2: mod_base = OTHER; note = 2 prime-O-methyl guanosine
  modified_base 3: mod_base = OTHER; note = 2 prime fluorouridine
  misc_feature 1..3: note = phosphorothioate linkage
  modified_base 4..5: mod_base = OTHER; note = 2 prime fluoroguanosine
  modified_base 6: mod_base = OTHER; note = 2 prime-O-methyl uridine
  modified_base 7: mod_base = OTHER; note = 2 prime-O-methyl uridine
  modified_base 8: mod_base = OTHER; note = 2 prime-O-methyl cytidine
  modified_base 9: mod_base = OTHER; note = 2 prime-O-methyl uridine
  modified_base 10: mod_base = OTHER; note = 2 prime-O-methyl adenosine
  modified_base 11..12: mod_base = OTHER; note = 2 prime fluorouridine
  modified_base 13: mod_base = OTHER; note = 2 prime-O-methyl adenosine
  modified_base 14: mod_base = OTHER; note = 2 prime-O-methyl uridine
  modified_base 15: mod_base = OTHER; note = 2 prime-O-methyl adenosine
  misc_feature 13^14: note = phosphorothioate linkage
  misc_feature 14^15: note = phosphorothioate linkage
tgtggttctattata

SEQ ID NO: 12 - RNA, 15 nt, synthetic construct
  source 1..15: mol_type = other RNA
  modified_base 1: mod_base = OTHER; note = 2 prime-O-methyl uridine
  modified_base 2: mod_base = OTHER; note = 2 prime-O-methyl guanosine
  modified_base 3: mod_base = OTHER; note = 2 prime fluorouridine
  misc_feature 1..3: note = phosphorothioate linkage
  modified_base 4: mod_base = OTHER; note = 2 prime-O-methyl guanosine
  modified_base 5: mod_base = OTHER; note = 2 prime fluoroguanosine
  modified_base 6: mod_base = OTHER; note = 2 prime-O-methyl uridine
  modified_base 7: mod_base = OTHER; note = 2 prime fluorouridine
  modified_base 8: mod_base = OTHER; note = 2 prime-O-methyl cytidine
  modified_base 9: mod_base = OTHER; note = 2 prime-O-methyl uridine
  modified_base 10: mod_base = OTHER; note = 2 prime-O-methyl adenosine
  modified_base 11: mod_base = OTHER; note = 2 prime fluorouridine
  modified_base 12: mod_base = OTHER; note = 2 prime-O-methyl uridine
  modified_base 13: mod_base = OTHER; note = 2 prime-O-methyl adenosine
  modified_base 14: mod_base = OTHER; note = 2 prime-O-methyl uridine
  modified_base 15: mod_base = OTHER; note = 2 prime-O-methyl adenosine
  misc_feature 13^14: note = phosphorothioate linkage
  misc_feature 14^15: note = phosphorothioate linkage
tgtggttctattata

(19) United States
(12) Patent Application Publication
(10) Pub. No.: US 2026/0097120 A1
(43) Pub. Date: Apr. 9, 2026

(54) INDUCTION OF STEM-LIKE ACTIVATED T CELLS

(71) Applicants: Phio Pharmaceuticals Corp., Marlborough, MA (US); Helmholtz Zentrum München Deutsches Forschungszentrum für Gesundheit und Umwelt (GMBH), Neuherberg (DE)

(72) Inventors: Simon Fricker, Marlborough, MA (US); James Cardia, Franklin, MA (US); Elfriede Noessner, Neuherberg (DE); Anna Bergmeier, Neuherberg (DE)

(73) Assignees: Phio Pharmaceuticals Corp., Marlborough, MA (US); Helmholtz Zentrum München Deutsches Forschungszentrum für Gesundheit und Umwelt (GMBH), Neuherberg (DE)

(21) Appl. No.: 19/113,774

(22) PCT Filed: Sep. 20, 2023
(86) PCT No.: PCT/US2023/074711

§ 371 Date: Mar. 20, 2025

Related U.S. Application Data
(60) Provisional application No. 63/408,802, filed on Sep. 21, 2022.

(51) Int. Cl. A61K 40/11; A61K 31/712; A61K 31/7125; A61K 31/713; A61K 35/17; A61K 40/31; A61K 40/32; A61K 40/36; A61K 40/42; C12N 5/0783; C12N 15/113

(52) U.S. Cl. CPC A61K 40/11; A61K 31/712; A61K 31/7125; A61K 31/713; A61K 35/17; A61K 40/31; A61K 40/32; A61K 40/36; A61K 40/42; C12N 5/0636; C12N 15/1138; C12N 2310/14; C12N 2310/315; C12N 2310/321

(57) ABSTRACT

The disclosure relates, in some aspects, to methods and compositions for production of immunogenic compositions. In some embodiments, the disclosure provides T cells which have been treated ex vivo with one or more oligonucleotide agents capable of controlling and/or reducing the differentiation of the T cell, for example through PD-1 silencing induced by chemically modified double stranded nucleic acid molecules. In some embodiments, compositions and methods described by the disclosure are useful as immunogenic modulators for treating cancer, such as melanoma.

### RELATED APPLICATION

This application claims the benefit under 35 U.S.C. § 119 (e) of U.S. Provisional Application No. 63/408,802, filed on Sep. 21, 2022, entitled “INDUCTION OF STEM-LIKE ACTIVATED T CELLS,” the entire disclosure of which is incorporated herein by reference in its entirety.

### REFERENCE TO AN ELECTRONIC SEQUENCE LISTING

The contents of the electronic sequence listing (R065970060WO00-SEQ-VLJ.xml; Size: 123,296 bytes; and Date of Creation: Sep. 13, 2023) is herein incorporated by reference in its entirety.

### FIELD

The disclosure pertains to the use nucleic acid molecules targeting programmed cell death receptor 1 (PD-1) to change the composition of a T cell population to take on a more stem-like phenotype and to the field of Adoptive Cell Transfer (ACT).

### BACKGROUND

A physiologic function of the immune system is to recognize and eliminate neoplastic cells. Therefore, an aspect of tumor progression is the development of immune resistance mechanisms. Once developed, these resistance mechanisms not only prevent the natural immune system from affecting the tumor growth, but also limit the efficacy of any immunotherapeutic approaches to cancer. An immune resistance mechanism involves immune-inhibitory pathways, sometimes referred to as immune checkpoints. The immune-inhibitory pathways play a particularly important role in the interaction between tumor cells and CD8+ cytotoxic T-lymphocytes, including Adoptive Cell Transfer (ACT) therapeutic agents.

Various methods of ACT involve ex vivo treatment of cells collected from a patient's samples, such as blood or tumor material. Common steps involved in the preparation of cell-based treatments are isolation of cells from the primary source (e.g., peripheral blood), gene editing (e.g., engineering of chimeric antigen receptor (CAR) T cells or engineered T cell receptor (TCR) cells), activation, and expansion.

During the ex vivo processing the cells undergo certain phenotypic changes that may affect their therapeutic properties, such as trafficking to the tumor, proliferative ability and longevity in vivo, and their efficacy in the immunosuppressive environment, among others. For example, the state of T-cell differentiation and maturation typically progresses through the following sequence of subtypes: naïve (T_{N})—stem cell memory (TSCM)—central memory (T_{CM}) —effector memory (T_{EM})—terminally differentiated effector T cells (T_{EFF}).

Immunotherapy of cancer has become increasingly important in clinical practice. Immunotherapies designed to elicit or amplify an immune response can be classified as activation immunotherapies, while immunotherapies that reduce or suppress immune response can be classified as suppression immunotherapies. One activation immunotherapeutic strategy to combat cancer immune resistance mechanisms is inhibiting immune checkpoints (e.g., by using checkpoint-targeting monoclonal antibodies) in order to stimulate or maintain a host immune response.

However, there are a number of drawbacks to using cancer immunotherapeutic agents in combination with checkpoint inhibitors. For example, immune checkpoint blockade can lead to the breaking of immune self-tolerance, thereby inducing a novel syndrome of autoimmune/auto-inflammatory side effects, designated “immune related adverse events.” Additionally, toxicity profiles of checkpoint inhibitors are reportedly different from the toxicity profiles reported for other classes of oncologic agents, and may induce inflammatory events in multiple organ systems, including skin, gastrointestinal, endocrine, pulmonary, hepatic, ocular, and the nervous system.

### SUMMARY

Described herein are T cells that are modified with nucleic acid molecules (e.g., INTASYL™ molecules) targeting programmed cell death protein 1 (PD-1).

In some aspects, the disclosure relates to compositions and methods for immunotherapy of diseases characterized by aberrant immune checkpoint function (e.g., cancer and certain infectious diseases). The disclosure is based, in part, on the discovery of immunomodulatory (e.g., immunogenic) compositions comprising a host cell (e.g., a T cell) comprising oligonucleotide molecules that target genes associated with tumor or infectious disease resistance mechanisms (e.g., PD-1) and methods of producing such compositions. As described herein, the compositions comprising a T cell comprising a nucleic acid molecule targeting PD-1 have been found to generate a T cell population having an increased proportion of stem-like CD8+ T cells. In some aspects, the disclosure provides chemically modified oligonucleotide molecules used in methods of producing immunogenic compositions. In some embodiments, methods and compositions described by the disclosure are useful for treating a subject having a proliferative or infectious disease.

Accordingly, in some aspects, the disclosure provides T cells modified with chemically modified double stranded nucleic acid molecules that target programmed cell death protein 1 (PD-1). In some embodiments, the modified T cells have a stem-like T cell phenotype (e.g., stem-like CD8+ T cells).

The disclosure, in some aspects, provides an immunogenic composition comprising a T cell comprising a chemically modified double stranded nucleic acid molecule that is directed against a programmed cell death 1 (PD-1) gene, wherein the chemically modified double stranded nucleic acid molecule comprises at least 12 contiguous nucleotides of a sequence selected from SEQ ID NOs: 1-12.

In some embodiments, the chemically modified double stranded nucleic acid molecule comprises a sequence selected from SEQ ID NOs: 1-12. In some embodiments, the chemically modified double stranded nucleic acid molecule is an INTASYL™. In some embodiments, the INTASYL™ is hydrophobically modified. In some embodiments, the INTASYL™ is linked to one or more hydrophobic conjugates. In some embodiments, the chemically modified double stranded nucleic acid molecule comprises at least one 2′-O-methyl modification and/or at least one 2′-O-Fluoro modification, and at least one phosphorothioate modification.

In some embodiments, the T cell comprises one or more transgenes expressing a chimeric antibody receptor (CAR). In some embodiments, the T cell comprises one or more T cell receptors (TCRs). In some embodiments, the TCR comprises a TCRaß heterodimer. In some embodiments, the T cell is a tumor infiltrating lymphocyte (TIL).

In some embodiments, the T cell is derived from a healthy donor.

In some embodiments, the chemically modified double stranded nucleic acid molecule induces at least 40% inhibition of PD-1 in the T cell. In some embodiments, the chemically modified double stranded nucleic acid molecule induces about 40% inhibition of PD-1 in the T cell.

In some embodiments, the chemically modified double stranded nucleic acid molecule comprises a sense strand having the sequence set forth in SEQ ID NO: 1 and/or an antisense strand having the sequence set forth in SEQ ID NO: 2 (PD1-1). In some embodiments, the chemically modified double stranded nucleic acid molecule comprises a sense strand having the sequence set forth in SEQ ID NO: 1 and/or antisense strand having the sequence set forth in SEQ ID NO: 3 (PD1-2). In some embodiments, the chemically modified double stranded nucleic acid molecule comprises a sense strand having the sequence set forth in SEQ ID NO: 1 and/or an antisense strand having the sequence set forth in SEQ ID NO: 4 (PD1-3). In some embodiments, the chemically modified double stranded nucleic acid molecule comprises a sense strand having the sequence set forth in SEQ ID NO: 1 and/or an antisense strand having the sequence set forth in SEQ ID NO: 5 (PD1-4). In some embodiments, the chemically modified double stranded nucleic acid molecule comprises a sense strand having the sequence set forth in SEQ ID NO: land/or an antisense strand having the sequence set forth in SEQ ID NO: 6 (PD1-5). In some embodiments, the chemically modified double stranded nucleic acid molecule comprises a sense strand having the sequence set forth in SEQ ID NO: 1 and/or an antisense strand having the sequence set forth in SEQ ID NO: 7 (PD1-6). In some embodiments, the chemically modified double stranded nucleic acid molecule comprises a sense strand having the sequence set forth in SEQ ID NO: 1 and/or an antisense strand having the sequence set forth in SEQ ID NO: 8 (PD1-7). In some embodiments, the chemically modified double stranded nucleic acid molecule comprises a sense strand having the sequence set forth in SEQ ID NO: 9 and/or an antisense strand having the sequence set forth in SEQ ID NO: 10 (PD1-8). In some embodiments, the chemically modified double stranded nucleic acid molecule comprises a sense strand having the sequence set forth in SEQ ID NO: 11 and/or an antisense strand having the sequence set forth in SEQ ID NO: 10 (PD1-9). In some embodiments, the chemically modified double stranded nucleic acid molecule comprises a sense strand having the sequence set forth in SEQ ID NO: 12 and/or an antisense strand having the sequence set forth in SEQ ID NO: 10 (PD1-10). In some embodiments, the chemically modified double stranded nucleic acid molecule comprises a sense strand having the sequence set forth in SEQ ID NO: 1 and/or an antisense strand having the sequence set forth in SEQ ID NO: 10 (PD1-11).

In some embodiments, the T cell is a CD8+ T cell. In some embodiments, the T cell has a stem-like T cell phenotype. In some embodiments, the T cell expresses T cell factor 1 (TCF-1). In some embodiments, the T cell further expresses CD95, CD45RA, CCR7, CD27, CD127, CD28, T-box expressed in T cells (T-bet), KI-67, thymocyte selection associated high mobility group box (TOX), or any combination thereof.

In some embodiments, the immunogenic composition further comprises a population of T cells and chemically modified double stranded nucleic acid molecules that are directed against a programmed cell death 1 (PD-1) gene, wherein the chemically modified double stranded molecules comprise at least 12 contiguous nucleotides of a sequence selected from SEQ ID NOs: 1-12.

In some embodiments, the population of T cells has a greater proportion of stem-like CD8+ T cells than an untreated population of T cells (e.g., a population of T cells that has not been exposed to any one of the chemically modified double stranded nucleic acid molecules described herein).

In some embodiments, the immunogenic composition comprises at least 5%, at least 10%, or at least 15% stem-like CD8+ T cells.

The disclosure, in some aspects, provides a method for producing a composition comprising stem-like T cells, the method comprising introducing one or more chemically modified double stranded nucleic molecules targeting PD-1 into T cells, wherein the chemically modified double stranded molecule comprises at least 12 contiguous nucleotides of a sequence selected from SEQ ID NOs: 1-12, thereby producing stem-like T cells.

In some embodiments, the stem-like T cells comprise stem-like CD8+ T cells.

In some embodiments, the chemically modified double stranded molecule comprises a sequence selected from SEQ ID NOs: 1-12.

In some embodiments, the chemically modified double stranded nucleic acid molecule is an INTASYL™.

In some embodiments, the chemically modified double stranded nucleic acid molecule comprises at least one 2′-O-methyl modification and/or at least one 2′-O-Fluoro modification, and at least one phosphorothioate modification.

In some embodiments, the chemically modified double stranded nucleic acid molecule induces at least 40% inhibition of PD-1 in the T cells. In some embodiments, the chemically modified double stranded nucleic acid molecule induces about 40% inhibition of PD-1 in the T cells.

In some embodiments, the chemically modified double stranded nucleic acid molecule comprises a sense strand having the sequence set forth in SEQ ID NO: 1 and/or an antisense strand having the sequence set forth in SEQ ID NO: 2 (PD1-1). In some embodiments, the chemically modified double stranded nucleic acid molecule comprises a sense strand having the sequence set forth in SEQ ID NO: 1 and/or antisense strand having the sequence set forth in SEQ ID NO: 3 (PD1-2). In some embodiments, the chemically modified double stranded nucleic acid molecule comprises a sense strand having the sequence set forth in SEQ ID NO: 1 and/or an antisense strand having the sequence set forth in SEQ ID NO: 4 (PD1-3). In some embodiments, the chemically modified double stranded nucleic acid molecule comprises a sense strand having the sequence set forth in SEQ ID NO: 1 and/or an antisense strand having the sequence set forth in SEQ ID NO: 5 (PD1-4). In some embodiments, the chemically modified double stranded nucleic acid molecule comprises a sense strand having the sequence set forth in SEQ ID NO: land/or an antisense strand having the sequence set forth in SEQ ID NO: 6 (PD1-5). In some embodiments, the chemically modified double stranded nucleic acid molecule comprises a sense strand having the sequence set forth in SEQ ID NO: 1 and/or an antisense strand having the sequence set forth in SEQ ID NO: 7 (PD1-6). In some embodiments, the chemically modified double stranded nucleic acid molecule comprises a sense strand having the sequence set forth in SEQ ID NO: 1 and/or an antisense strand having the sequence set forth in SEQ ID NO: 8 (PD1-7). In some embodiments, the chemically modified double stranded nucleic acid molecule comprises a sense strand having the sequence set forth in SEQ ID NO: 9 and/or an antisense strand having the sequence set forth in SEQ ID NO: 10 (PD1-8). In some embodiments, the chemically modified double stranded nucleic acid molecule comprises a sense strand having the sequence set forth in SEQ ID NO: 11 and/or an antisense strand having the sequence set forth in SEQ ID NO: 10 (PD1-9). In some embodiments, the chemically modified double stranded nucleic acid molecule comprises a sense strand having the sequence set forth in SEQ ID NO: 12 and/or an antisense strand having the sequence set forth in SEQ ID NO: 10 (PD1-10).

In some embodiments, the T cell expresses T cell factor 1 (TCF-1). In some embodiments, the T cell further expresses CD95, CD45RA, CCR7, CD27, CD127, CD28, T-bet, KI-67, TOX, or any combination thereof.

The disclosure, in some aspects, provides a method comprising introducing into a T cell ex vivo a chemically modified double stranded nucleic acid molecule that is directed against a programmed cell death 1 (PD-1) gene, wherein the chemically modified double stranded nucleic acid molecule comprises at least 12 contiguous nucleotides of a sequence selected from SEQ ID NOs: 1-12.

The disclosure, in some embodiments, provides a method for treating a subject suffering from a proliferative disease, the method comprising administering to the subject any one of the immunogenic compositions described herein.

In some embodiments, the proliferative disease is cancer.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIG. 1 shows the composition of TCR53-T cell populations without treatment (untreated control, “UTC”) or incubated with a PD-1-targeting INTASYL™ compound (“siPD-1”) for 24 hours, followed by co-culture with an autologous tumor cell line for 96 hours (“96 h”).

### DETAILED DESCRIPTION

Adoptive cell transfer (ACT) of T cells is a powerful tool in the treatment of cancer. However, there are still hurdles to satisfactory efficacy, including the upregulation of immune-inhibitor receptors like PD-1, as well as poor persistence of effector T cells in patients. Importantly, the T cell differentiation state is closely link to the T cell's functional capacity, controlling effector activity, memory, and renewal capacity. Transcription factor expression closely regulates the path along which the T cell progresses during antigenic stimulation. Stem-like memory subsets of CD8+ T cells, such as those marked by T cell factor 1 (TCF-1) expression, may therefore represent an advantageous effector population for ACT, as they show longer persistence, higher proliferative activity, responsiveness to checkpoint inhibitors, and the ability to differentiate into new effector T cells (e.g., cytotoxic effector T cells).

As described herein, it was found that silencing PD-1 changed the composition of a T cell population that developed during antigenic co-culture, such that a greater proportion of the T cell population possessed stem-like characteristics (e.g., is TCF-1-positive).

Thus, this disclosure, in some aspects, relates to compositions and methods for immunotherapy. The disclosure is based, in part, on chemically modified double stranded nucleic acid molecules (e.g., INTASYL™) targeting PD-1, which is associated with controlling the differentiation process of T cells and/or modulation of T cell expression or activity. INTASYL™ technology is particularly suitable for controlling the differentiation process of cells, including T cells, and the production of therapeutic cells rich in the desired subtypes (e.g., stem-like T cells).

As immune cells are dysregulated in a tumor suppressive microenvironment, immunomodulatory monoclonal antibodies (mAbs) have been applied to block the inhibitory signals or activate the co-stimulatory pathway, thereby aiming to enhance the persistence and activity of immune cells. Immunomodulatory mAbs interact with soluble or cellular components of the immune system. Blockade of immunosuppressive receptors expressed on natural killer (NK) cells or T cells (also known as checkpoint blockade mAbs), such anti-PD1 antibodies, has become a mainstream treatment and are now even offered as first line treatment. Still, only a limited number of patients react well to these drugs, resulting in complete remissions and extended survival and cure.

Cellular immunotherapies are novel treatment methods that boost or extend the immune system to eliminate cancer. ACT is a “living cell” drug remedy that involves the procedure of obtaining anti-tumor effector cells (mostly T cell) from a patient (autologous) or from a donor (allogeneic), followed by expansion and/or engineering these effector cells in vitro and infusion of such cells into patients suffering from malignant diseases.

ACT was first reported in 1988, which used tumor-infiltrating lymphocytes (TIL) administered to melanoma patients and achieved complete regression (CR) in some patients. Cytotoxic T cells are found to be potent tumor killer cells that act by recognizing specific tumor antigens by TCR. This approach has been used in engineering antigen specific T cells and more recently creating chimeric antigen receptor T cells (CAR-T) very active in B-cell leukemias.

In vitro activation of the effector cells allows these cells to be released from the suppressive microenvironment existing in the tumor, boosts their cytotoxicity, and leads to their expansion. During the expansion period, effector cells can be further tailored or customized towards specific tumors by genetic modification methods, including but not limited to gene editing, such as by CRISPR/Cas9, viral transduction, and mRNA transfection.

However, as severe side effects have been reported with CAR-T, including Cytokine Release Syndrome, Immune effector Cell-Associated Neurotoxicity Syndrome, and cytopenia, novel therapies with fewer side effects are urgently required for ACT in tumor immunology.

Aspects of the present disclosure relate to modification of T cells with self-delivering RNA interference (RNAi) compounds, termed INTASYL™. The use of INTASYL™ presents advantages relative to previous approaches as these compounds are rapidly and efficiently taken up by T cells, result in transfection of ˜100% of the T cells while maintaining high viability, are easily incorporated into the manufacturing process, and have a lower cost of goods than genetic modification.

INTASYL™ technology is particularly suitable for modulating genes of interest in T cells and results in enhanced T cell activity. Several advantages of INTASYL™ include: (i) INTASYL™ can be developed in a short period of time and can silence virtually any target including “non-druggable” targets, e.g., those that are difficult to inhibit by small molecules, e.g., transcription factors; (ii) compared to alternative ex vivo siRNA transfection techniques (e.g., lipid mediated transfection or electroporation), INTASYL™ can transfect a variety of cell types, including T cells with high transfection efficiency retaining a high cell viability; (iii) when added to cell culture media at an early expansion stage, INTASYL™ compounds provide transient silencing of targets of interest during 8-10 division cycles; (iv) INTASYL™ can be used in combination to simultaneously silence multiple targets, thus providing considerable flexibility for the use in different types of cell treatment protocols.

Described herein are INTASYL™ compounds directed to specific targets involved in the differentiation of T cells, and the beneficial effect of such INTASYL™ compounds on the phenotype of T cells following ex vivo expansion.

As used herein, “nucleic acid molecule” includes but is not limited to: INTASYL™ compounds, sd-rxRNA, rxRNAori, oligonucleotides, ASO, siRNA, shRNA, miRNA, ncRNA, cp-lasiRNA, aiRNA, single stranded nucleic acid molecules, double stranded nucleic acid molecules, RNA and DNA. In some embodiments, the nucleic acid molecule is a chemically modified nucleic acid molecule, such as a chemically modified oligonucleotide. In some embodiments, the nucleic acid molecule is double stranded. In some embodiments, chemically modified double stranded nucleic acid molecules as described herein are sd-rxRNA or INTASYL™ compounds.

INTASYL™ (sd-rxRNA) Molecules

Aspects of the disclosure relate to INTASYL™ compounds that target genes associated with controlling the differentiation process of T cells and/or modulating T cell expression or activity, such as PD-1. In some embodiments, the disclosure provides an INTASYL™ compound targeting PD-1 (PDCD1). In some embodiments, an INTASYL™ compound described herein comprises or consists of, or is targeted to or directed against, a sequence set forth in Table 1, or a fragment thereof.

As used herein, an “sd-rxRNA” or an “sd-rxRNA molecule” or an “INTASYL™” or an “INTASYL™ molecule” or an “INTASYL™ compound,” which are all used interchangeably, refers to a self-delivering RNA molecule such as those described in, and incorporated by reference from, U.S. Pat. No. 8,796,443, granted on Aug. 5, 2014, entitled “REDUCED SIZE SELF-DELIVERING RNAI COMPOUNDS”, U.S. Pat. No. 9,080,171, granted on Jul. 14, 2015, entitled “REDUCED SIZE SELF-DELIVERING RNAI COMPOUNDS”, U.S. Pat. No. 9,175,289, granted on Nov. 3, 2015, entitled “REDUCED SIZE SELF-DELIVERING RNAI COMPOUNDS”, U.S. Pat. No. 10,240,149, granted on Mar. 26, 2019, entitled “REDUCED SIZE SELF-DELIVERING RNAI COMPOUNDS”, U.S. Pat. No. 10,774,330, granted on Sep. 15, 2020, entitled “REDUCED SIZE SELF-DELIVERING RNAI COMPOUNDS”, U.S. Pat. No. 11,118,178, granted on Sep. 14, 2021, entitled “REDUCED SIZE SELF-DELIVERING RNAI COMPOUNDS”, PCT Publication No. WO2010/033247 (Application No. PCT/US2009/005247), filed on Sep. 22, 2009, and entitled “REDUCED SIZE SELF-DELIVERING RNAI COMPOUNDS”, and PCT Publication No. WO2011/119852 (Application No. PCT/US2011/029824), filed on Mar. 24, 2011, and entitled “REDUCED SIZE SELF-DELIVERING RNAI COMPOUNDS.” Briefly, an INTASYL™, (also referred to as an sd-rxRNA or an sd-rxRNAnano) is an isolated asymmetric double stranded nucleic acid molecule comprising a guide strand, with a minimal length of 16 nucleotides, and a passenger strand of 8-18 nucleotides in length, wherein the double stranded nucleic acid molecule has a double stranded region and a single stranded region, the single stranded region having 4-12 nucleotides in length and having at least three nucleotide backbone modifications. In preferred embodiments, the double stranded nucleic acid molecule has one end that is blunt or includes a one or two nucleotide overhang. INTASYL™ molecules can be optimized through chemical modification, and in some instances through attachment of hydrophobic conjugates. Each of the above-referenced patents and publications are incorporated by reference herein in their entireties.

In some embodiments, an INTASYL™ comprises an isolated double stranded nucleic acid molecule comprising a guide strand and a passenger strand, wherein the region of the molecule that is double stranded is from 8-15 nucleotides long, wherein the guide strand contains a single stranded region that is 4-12 nucleotides long, wherein the single stranded region of the guide strand contains 3, 4, 5, 6, 7, 8, 9, 10, 11 or 12 phosphorothioate modifications, and wherein at least 40% of the nucleotides of the double stranded nucleic acid are modified.

In some embodiments, an INTASYL™ comprises an isolated double stranded nucleic acid molecule comprising a guide strand and a passenger strand, wherein the region of the molecule that is double stranded is from 8-15 nucleotides long, wherein the guide strand contains a single stranded region that is 4-12 nucleotides long, wherein the single stranded region of the guide strand contains 3, 4, 5, 6, 7, 8, 9, 10, 11 or 12 phosphorothioate modifications, and wherein at least 40% of the nucleotides of the double stranded nucleic acid are modified, wherein one strand, is conjugated to cholesterol at the 5′ or 3′ end of the strand. In some embodiments, the passenger strand is conjugated cholesterol at the 5′ or 3′ end of the strand.

In some embodiments, an INTASYL™ comprises an isolated double stranded nucleic acid molecule comprising a guide strand and a passenger strand, wherein the region of the molecule that is double stranded is from 8-15 nucleotides long, wherein the guide strand contains a single stranded region that is 4-12 nucleotides long, wherein the single stranded region of the guide strand contains 3, 4, 5, 6, 7, 8, 9, 10, 11 or 12 phosphorothioate modifications, and wherein at least 40% of the nucleotides of the double stranded nucleic acid are modified, wherein one strand, e.g., the passenger strand, contains a hydrophobic moiety conjugated to the 3′ end of the passenger strand, wherein the hydrophobic moiety is cholesterol.

Nucleic acid molecules associated with the disclosure include isolated double stranded or duplex nucleic acids, chemically modified double stranded or duplex nucleic acids, oligonucleotides, polynucleotides, nano molecules, nano RNA, sd-rxRNA1ano, sd-rxRNA, and INTASYL™

INTASYL™ molecules are much more effectively taken up by cells compared to conventional siRNAs. These molecules are highly efficient in silencing of target gene expression and offer significant advantages over previously described RNAi molecules including high activity in the presence of serum, efficient self-delivery, compatibility with a wide variety of linkers, and reduced presence or complete absence of chemical modifications that are associated with toxicity.

In contrast to single stranded polynucleotides, duplex polynucleotides have traditionally been difficult to deliver to a cell as they have rigid structures and a large number of negative charges, which makes membrane transfer difficult. INTASYL™ molecules however, although partially double stranded, are recognized in vivo as single stranded and, as such, are capable of efficiently being delivered across cell membranes. As a result, the polynucleotides of the disclosure are capable in many instances of self-delivery. Thus, the polynucleotides of the disclosure may be formulated in a manner similar to conventional RNAi agents or they may be delivered to the cell or subject alone (or with non-delivery type carriers) and allowed to self-deliver. In one embodiment of the present disclosure, self-delivering asymmetric double stranded RNA molecules are provided in which one portion of the molecule resembles a conventional RNA duplex and a second portion of the molecule is single stranded.

The oligonucleotides of the disclosure in some aspects have a combination of asymmetric structures including a double stranded region and a single stranded region of 5 nucleotides or longer, specific chemical modification patterns and are conjugated to lipophilic or hydrophobic molecules. In some embodiments, this class of RNAi like compounds have superior efficacy in vitro and in vivo. It is believed that the reduction in the size of the rigid duplex region in combination with phosphorothioate modifications applied to a single stranded region contribute to the observed superior efficacy.

In some embodiments, the RNAi compounds of the disclosure comprise an asymmetric compound comprising a duplex region (required for efficient RISC entry) of 8-15 bases long and a single stranded region of 4-12 nucleotides long. In some embodiments, the duplex region is 13 or 14 nucleotides long, and in some embodiments, the since stranded region is 6-7 nucleotides long. The single stranded region of the RNAi compounds (e.g., INTASYL™ molecules) also comprises 2-12 phosphorothioate internucleotide linkages (referred to as phosphorothioate modifications). In some embodiments, the single stranded region comprises 6-8 phosphorothioate internucleotide linkages. Additionally, the RNAi compounds of the disclosure also include a unique chemical modification pattern, which provides stability and is compatible with RISC entry. In some embodiments, the combination of these elements has resulted in unexpected properties, which are highly useful for delivery of RNAi reagents in vitro and in vivo.

The chemical modification pattern, which provides stability and is compatible with RISC entry can include modifications to the sense, or passenger, strand as well as the antisense, or guide, strand. For instance, the passenger strand can be modified with any chemical entities, which confirm stability and do not interfere with activity. Such modifications include 2′ ribo modifications (O-methyl, 2′ F, 2 deoxy and others) and backbone modifications, such as phosphorothioate modifications. In some embodiments, the chemical modification pattern in the passenger strand includes O-methyl modification of C and U nucleotides within the passenger strand or alternatively, the passenger strand may be completely O-methyl modified.

The guide strand, in some embodiments, may also be modified by any chemical modification which confirms stability without interfering with RISC entry. In some embodiments, the chemical modification pattern in the guide strand includes the majority of C and U nucleotides being 2′ F modified and the 5′ end being phosphorylated. In some embodiments, a chemical modification pattern in the guide strand includes 2′O-methyl modification of position 1 and C/U in positions 11-18 and 5′ end chemical phosphorylation. In some embodiments, a chemical modification pattern in the guide strand includes 2′O-methyl modification of position 1 and C/U in positions 11-18 and 5′ end chemical phosphorylation and 2′F modification of C/U in positions 2-10. In some embodiments, the passenger strand and/or the guide strand contains at least one 5-methyl C or U modification.

In some embodiments, at least 30% of the nucleotides in the sd-rxRNA (e.g., INTASYL™ compound) are modified. For example, in some embodiments, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the INTASYL™ compound are modified. In some embodiments, 100% of the nucleotides in the INTASYL™ compound are modified.

The above-described chemical modification patterns of the oligonucleotides of the disclosure are well tolerated and improve efficacy of asymmetric RNAi compounds. In some embodiments, elimination of any of the described components (guide strand stabilization, phosphorothioate stretch, sense strand stabilization, hydrophobic conjugate and/or targeting ligand) or increase in size, can result in sub-optimal efficacy and, in some instances, complete loss of efficacy. The combination of elements results in development of a compound, which is fully active following passive delivery to cells.

The INTASYL™ can be further improved in some instances by improving the hydrophobicity of compounds using novel types of chemistries. For example, one chemistry is related to use of hydrophobic base modifications. Any base in any position might be modified, as long as modification results in an increase of the partition coefficient of the base. The preferred locations for modification chemistries are positions 4 and 5 of the pyrimidines. The major advantage of these positions is (a) ease of synthesis and (b) lack of interference with base-pairing and A form helix formation, which are essential for RISC complex loading and target recognition. In some embodiments, INTASYL™ compounds where multiple deoxy uridines are present without interfering with overall compound efficacy are used. In addition, major improvement in tissue distribution and cellular uptake might be obtained by modifying the structure of the hydrophobic conjugate. In some embodiments, the structure of sterol is modified to alter (increase/decrease) C17 attached chain. This type of modification results in significant increase in cellular uptake and improvement of tissue uptake prosperities in vivo.

In some embodiments, a chemically modified double stranded nucleic acid molecule is a hydrophobically modified siRNA-antisense hybrid molecule, comprising a double stranded region of about 13-22 base pairs, with or without a 3′-overhang on each of the sense and antisense strands, and a 3′ single stranded tail on the antisense strand of about 2-9 nucleotides. In some embodiments, the chemically modified double stranded nucleic acid molecule contains at least one 2′-O-Methyl modification, at least one 2′-Fluoro modification, and at least one phosphorothioate modification, as well as at least one hydrophobic modification selected from sterol, cholesterol, vitamin D, napthyl, isobutyl, benzyl, indol, tryptophane, phenyl, and the like hydrophobic modifiers. In some embodiments, a chemically modified double stranded nucleic acid molecule comprises a plurality of such modifications.

In some aspects, the disclosure relates to chemically modified double stranded nucleic acid molecules that target genes encoding targets related to differentiation of cells (e.g., differentiation of T-cells), such as signal transduction/transcription factor targets, epigenetic targets, metabolic and co-inhibitory/negative regulatory targets.

In some aspects, the disclosure relates to chemically modified double stranded nucleic acid molecules that target genes encoding immune checkpoint proteins. Generally, an immune checkpoint protein is a protein that modulates a host immune response (e.g., by stimulating or suppressing T cell function). In some embodiments, a chemically modified double stranded nucleic acid molecule targets a gene encoding PD-1.

As used herein, “PDCD1” or “PD1” refers to Programmed Cell Death Protein 1, which is a cell surface receptor that functions to down-regulate the immune system and promote immune self-tolerance by suppressing T-cell-mediated inflammatory activity. In some embodiments, PDCD1 is encoded by a nucleic acid sequence represented by NCBI Reference Sequence Number NM_005018.2. Non-limiting examples of PD-1 sequences that may be targeted by chemically modified double stranded nucleic acid molecules of the disclosure are listed in Table 1. In some embodiments, a chemically modified double stranded nucleic acid molecule, such as an INTASYL™, targets any one of SEQ ID NOs: 1-12 or a portion thereof.

In some embodiments a chemically modified double stranded nucleic acid molecule, such as an INTASYL™, comprises at least 12 contiguous nucleotides of a sequence within Table 1. In some embodiments, a chemically modified double stranded nucleic acid molecule, such as an INTASYL™, comprises at least one sequence within Table 1. In some embodiments a chemically modified double stranded nucleic acid molecule, such as an INTASYL™, comprises at least 12 contiguous nucleotides of a sequence selected from SEQ ID NOs: 1-12. In some embodiments, a chemically modified double stranded nucleic acid molecule, such as an INTASYL™, comprises a sequence selected from SEQ ID NOs: 1-12.

In some embodiments, a chemically modified double stranded nucleic acid molecule comprises a sense strand having the sequence set forth in SEQ ID NO: 1 and/or an antisense strand having the sequence set forth in SEQ ID NO: 2 (PD1-1). In some embodiments, a chemically modified double stranded nucleic acid molecule (e.g., an INTASYL™ molecule) comprises a sense strand having the sequence set forth in SEQ ID NO: 1 and an antisense strand having the sequence set forth in SEQ ID NO: 3 (PD1-2). In some embodiments, a chemically modified double stranded nucleic acid molecule comprises a sense strand having the sequence set forth in SEQ ID NO: 1 and/or an antisense strand having the sequence set forth in SEQ ID NO: 4 (PD1-3). In some embodiments, a chemically modified double stranded nucleic acid molecule comprises a sense strand having the sequence set forth in SEQ ID NO: 1 and/or an antisense strand having the sequence set forth in SEQ ID NO: 5 (PD1-4). In some embodiments, a chemically modified double stranded nucleic acid molecule comprises a sense strand having the sequence set forth in SEQ ID NO: 1 and/or an antisense strand having the sequence set forth in SEQ ID NO: 6 (PD1-5). In some embodiments, a chemically modified double stranded nucleic acid molecule comprises a sense strand having the sequence set forth in SEQ ID NO: 1 and/or an antisense strand having the sequence set forth in SEQ ID NO: 7 (PD1-6). In some embodiments, a chemically modified double stranded nucleic acid molecule comprises a sense strand having the sequence set forth in SEQ ID NO: 1 and/or an antisense strand having the sequence set forth in SEQ ID NO: 8 (PD1-7). In some embodiments, a chemically modified double stranded nucleic acid molecule comprises a sense strand having the sequence set forth in SEQ ID NO: 9 and/or an antisense strand having the sequence set forth in SEQ ID NO: 10 (PD1-8). In some embodiments, a chemically modified double stranded nucleic acid molecule comprises a sense strand having the sequence set forth in SEQ ID NO: 11 and/or an antisense strand having the sequence set forth in SEQ ID NO: 10 (PD1-9). In some embodiments, a chemically modified double stranded nucleic acid molecule comprises a sense strand having the sequence set forth in SEQ ID NO: 12 and/or an antisense strand having the sequence set forth in SEQ ID NO: 10 (PD1-10).

Thus, aspects of the disclosure relate to isolated double stranded nucleic acid molecules comprising a guide (antisense) strand and a passenger (sense) strand. As used herein, the term “double stranded” refers to one or more nucleic acid molecules in which at least a portion of the nucleomonomers is complementary and hydrogen bond to form a double stranded region. In some embodiments, the length of the guide strand ranges from 16-29 nucleotides long. In certain embodiments, the guide strand is 16, 17, 18, 19, 20, 21, 22, 23, 24, 25, 26, 27, 28, or 29 nucleotides long. The guide strand has complementarity to a target gene. Complementarity between the guide strand and the target gene may exist over any portion of the guide strand. Complementarity as used herein may be perfect complementarity or less than perfect complementarity as long as the guide strand is sufficiently complementary to the target that it mediates RNAi. In some embodiments complementarity refers to less than 25%, 20%, 15%, 10%, 5%, 4%, 3%, 2%, or 1% mismatch between the guide strand and the target. Perfect complementarity refers to 100% complementarity. In some embodiments, siRNA sequences with insertions, deletions, and single point mutations relative to the target sequence have also been found to be effective for inhibition. Moreover, not all positions of a siRNA contribute equally to target recognition. Mismatches in the center of the siRNA are most critical and essentially abolish target RNA cleavage. Mismatches upstream of the center or upstream of the cleavage site referencing the antisense strand are tolerated but significantly reduce target RNA cleavage. Mismatches downstream of the center or cleavage site referencing the antisense strand, preferably located near the 3′ end of the antisense strand, e.g., 1, 2, 3, 4, 5 or 6 nucleotides from the 3′ end of the antisense strand, are tolerated and reduce target RNA cleavage only slightly.

While not wishing to be bound by any particular theory, in some embodiments of double stranded nucleic acid molecules described herein, the guide strand is at least 16 nucleotides in length and anchors the Argonaute protein in RISC. In some embodiments, when the guide strand loads into RISC it has a defined seed region and target mRNA cleavage takes place across from position 10-11 of the guide strand. In some embodiments, the 5′ end of the guide strand is or is able to be phosphorylated. The nucleic acid molecules described herein may be referred to as minimum trigger RNA.

In some embodiments of double stranded nucleic acid molecules described herein, the length of the passenger strand ranges from 8-15 nucleotides long. In some embodiments of double stranded nucleic acid molecules described herein, the length of the passenger strand ranges from 8-16 nucleotides long. In certain embodiments, the passenger strand is 8, 9, 10, 11, 12, 13, 14, 15, or 16 nucleotides long. The passenger strand has complementarity to the guide strand. Complementarity between the passenger strand and the guide strand can exist over any portion of the passenger or guide strand. In some embodiments, there is 100% complementarity between the guide and passenger strands within the double stranded region of the molecule.

Aspects of the disclosure relate to double stranded nucleic acid molecules with minimal double stranded regions. In some embodiments the region of the molecule that is double stranded ranges from 8-15 nucleotides long. In some embodiments the region of the molecule that is double stranded ranges from 8-16 nucleotides long. In certain embodiments, the region of the molecule that is double stranded is 8, 9, 10, 11, 12, 13, 14, 15, or 16 nucleotides long. In certain embodiments the double stranded region is 13 or 14 nucleotides long. In some embodiments, the region of the molecule that is double stranded is 13-22 nucleotides long. In certain embodiments, the region of the molecule that is double stranded is 16, 17, 18, 19, 20, 21 or 22 nucleotides long.

There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. For example, the single stranded region can be 4, 5, 6, 7, 8, 9, 10, 11 or 12 nucleotides long. However, in certain embodiments, the single stranded region can also be less than 4 or greater than 12 nucleotides long. In certain embodiments, the single stranded region is at least 6 or at least 7 nucleotides long. In some embodiments, the single stranded region is 2-9 nucleotides long, including 2 or 3 nucleotides long.

RNAi constructs associated with the disclosure can have a thermodynamic stability (ΔG) of less than-13 kkal/mol. In some embodiments, the thermodynamic stability (ΔG) is less than-20 kkal/mol. In some embodiments there is a loss of efficacy when (ΔG) goes below-21 kkal/mol. In some embodiments a (ΔG) value higher than −13 kkal/mol is compatible with aspects of the disclosure. Without wishing to be bound by any theory, in some embodiments a molecule with a relatively higher (ΔG) value may become active at a relatively higher concentration, while a molecule with a relatively lower (ΔG) value may become active at a relatively lower concentration. In some embodiments, the (ΔG) value may be higher than-9 kkcal/mol. The gene silencing effects mediated by the RNAi constructs associated with the disclosure, containing minimal double stranded regions, are unexpected because molecules of almost identical design but lower thermodynamic stability have been demonstrated to be inactive (Rana et al 2004).

Without wishing to be bound by any theory, results described herein suggest that a stretch of 8-10 bp of dsRNA or dsDNA will be structurally recognized by protein components of RISC or co-factors of RISC. Additionally, there is a free energy requirement for the triggering compound that it may be either sensed by the protein components and/or stable enough to interact with such components so that it may be loaded into the Argonaute protein. If acceptable thermodynamics are present and there is a double stranded portion that is preferably at least 8 nucleotides, then the duplex will be recognized and loaded into the RNAi machinery.

In some embodiments, thermodynamic stability is increased through the use of LNA bases. In some embodiments, additional chemical modifications are introduced. Several non-limiting examples of chemical modifications include: 5′ Phosphate, 5′Phosphonate, 5′ Vinyl Phosphonate, 2′-O-methyl, 2′-O-ethyl, 2′-fluoro, ribothymidine, C-5 propynyl-dC (pdC) and C-5 propynyl-dU (pdU); C-5 propynyl-C(pC) and C-5 propynyl-U (pU); 5-methyl C, 5-methyl U, 5-methyl dC, 5-methyl dU methoxy, (2,6-diaminopurine), 5′-Dimethoxytrityl-N4-ethyl-2′-deoxyCytidine and MGB (minor groove binder). It should be appreciated that more than one chemical modification can be combined within the same molecule.

Molecules associated with the disclosure are optimized for increased potency and/or reduced toxicity. For example, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. Specifically, reduction in 2′F content of a molecule is predicted to reduce toxicity of the molecule. Furthermore, the number of phosphorothioate modifications in an RNA molecule can influence the uptake of the molecule into a cell, for example the efficiency of passive uptake of the molecule into a cell. Preferred embodiments of molecules described herein have no 2′F modification and yet are characterized by equal efficacy in cellular uptake and tissue penetration. Such molecules represent a significant improvement over prior art, such as molecules described by Accell and Wolfrum, which are heavily modified with extensive use of 2′F.

In some embodiments, a guide strand is approximately 18-20 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contain 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated and/or contains a vinyl phosphonate. C and U nucleotides within the guide strand can be 2′F modified. For example, C and U nucleotides in positions 2-10 of a 20 nucleotide guide strand (or corresponding positions in a guide strand of a different length) can be 2′F modified. C and U nucleotides within the guide strand can also be 2′OMe modified. For example, C and U nucleotides in positions 11-18 of a 19 nucleotide guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

In some aspects, a passenger strand is approximately 11-14 nucleotides in length. The passenger strand may contain modifications that confer increased stability. One or more nucleotides in the passenger strand can be 2′OMe modified. In some embodiments, one or more of the C and/or U nucleotides in the passenger strand is 2′OMe modified, or all of the C and U nucleotides in the passenger strand are 2′OMe modified. In certain embodiments, all of the nucleotides in the passenger strand are 2′OMe modified. One or more of the nucleotides on the passenger strand can also be phosphate-modified such as phosphorothioate modified. The passenger strand can also contain 2′ ribo, 2′F and 2 deoxy modifications or any combination of the above. Chemical modification patterns on both the guide and passenger strand can be well tolerated and a combination of chemical modifications can lead to increased efficacy and self-delivery of RNA molecules.

Aspects of the disclosure relate to RNAi constructs that have extended single stranded regions relative to double stranded regions, as compared to molecules that have been used previously for RNAi. The single stranded region of the molecules may be modified to promote cellular uptake or gene silencing. In some embodiments, phosphorothioate modification of the single stranded region influences cellular uptake and/or gene silencing. The region of the guide strand that is phosphorothioate modified can include nucleotides within both the single stranded and double stranded regions of the molecule. In some embodiments, the single stranded region includes 2-12 phosphorothioate modifications. For example, the single stranded region can include 2, 3, 4, 5, 6, 7, 8, 9, 10, 11, or 12 phosphorothioate modifications. In some instances, the single stranded region contains 6-8 phosphorothioate modifications.

Molecules associated with the disclosure are also designed for cellular uptake. In RNA molecules described herein, the guide and/or passenger strands can be attached to a conjugate. In certain embodiments the conjugate is hydrophobic. The hydrophobic conjugate can be a small molecule with a partition coefficient that is higher than 10. The conjugate can be a sterol-type molecule such as cholesterol, or a molecule with an increased length polycarbon chain attached to C17, and the presence of a conjugate can influence the ability of an RNA molecule to be taken into a cell with or without a lipid transfection reagent. The conjugate can be attached to the passenger or guide strand through a hydrophobic linker. In some embodiments, a hydrophobic linker is 5-12C in length, and/or is hydroxypyrrolidine-based. In some embodiments, a hydrophobic conjugate is attached to the passenger strand and the CU residues of either the passenger and/or guide strand are modified. In some embodiments, at least 50%, 55%, 60%, 65%, 70%, 75%, 80%, 85%, 90% or 95% of the CU residues on the passenger strand and/or the guide strand are modified. In some aspects, molecules associated with the disclosure are self-delivering (sd). As used herein, “self-delivery” refers to the ability of a molecule to be delivered into a cell without the need for an additional delivery vehicle such as a transfection reagent.

In some embodiments, molecules associated with the disclosure are designed for targeted delivery to the liver. In RNA molecules described herein, the guide and/or passenger strands can be attached to a conjugate. In certain embodiments the conjugate is a targeting ligand. The targeting ligand conjugate can be a saccharide such as N-acetyl galactosamine (GalNac) moieties and derivatives thereof. The RNA molecules, in some embodiments, may comprise 1, 2, 3, 4, 5 or more GalNac moieties. The targeting ligand conjugate(s) can be attached to the passenger or guide strand through a linker or incorporated into the passenger or guide strand as a phosphoramidite, for example.

Aspects of the disclosure relate to selecting molecules for use in RNAi. In some embodiments, molecules that have a double stranded region of 8-15 nucleotides can be selected for use in RNAi. In some embodiments, molecules are selected based on their thermodynamic stability (ΔG). In some embodiments, molecules will be selected that have a (ΔG) of less than-13 kkal/mol. For example, the (ΔG) value may be −13, −14, −15, −16, −17, −18, −19, −21, −22 or less than-22 kkal/mol. In other embodiments, the (ΔG) value may be higher than-13 kkal/mol. For example, the (ΔG) value may be −12, −11, −10, −9, −8, −7 or more than −7 kkal/mol. It should be appreciated that ΔG can be calculated using any method known in the art. In some embodiments ΔG is calculated using Mfold, available through the Mfold internet site (mfold.bioinfo.rpi.edu/cgi-bin/rna-form1.cgi). Methods for calculating ΔG are described in, and are incorporated by reference from, the following references: Zuker, M. (2003) Nucleic Acids Res., 31 (13): 3406-15; Mathews, D. H., Sabina, J., Zuker, M. and Turner, D. H. (1999) J. Mol. Biol. 288:911-940; Mathews, D. H., Disney, M. D., Childs, J. L., Schroeder, S. J., Zuker, M., and Turner, D. H. (2004) Proc. Natl. Acad. Sci. 101:7287-7292; Duan, S., Mathews, D. H., and Turner, D. H. (2006) Biochemistry 45:9819-9832; Wuchty, S., Fontana, W., Hofacker, I. L., and Schuster, P. (1999) Biopolymers 49:145-165.

In certain embodiments, the polynucleotide contains 5′- and/or 3′-end overhangs. The number and/or sequence of nucleotides overhang on one end of the polynucleotide may be the same or different from the other end of the polynucleotide. In certain embodiments, one or more of the overhang nucleotides may contain chemical modification(s), such as phosphorothioate or 2′-OMe modification.

In certain embodiments, the polynucleotide is unmodified. In other embodiments, at least one nucleotide is modified. In further embodiments, the modification includes a 2′-H or 2′-modified ribose sugar at the 2nd nucleotide from the 5′-end of the guide sequence. The “2nd nucleotide” is defined as the second nucleotide from the 5′-end of the polynucleotide.

As used herein, “2′-modified ribose sugar” includes those ribose sugars that do not have a 2′—OH group. “2′-modified ribose sugar” does not include 2′-deoxyribose (found in unmodified canonical DNA nucleotides). For example, the 2′-modified ribose sugar may be 2′-O-alkyl nucleotides, 2′-deoxy-2′-fluoro nucleotides, 2′-deoxy nucleotides, or combination thereof.

In certain embodiments, the 2′-modified nucleotides are pyrimidine nucleotides (e.g., C/U). Examples of 2′-O-alkyl nucleotides include 2′-O-methyl nucleotides, or 2′-O-allyl nucleotides.

In certain embodiments, the sd-rxRNA polynucleotide of the disclosure with the above-referenced 5′-end modification exhibits significantly (e.g., at least about 25%, 30%, 35%, 40%, 45%, 50%, 55%, 60%, 65%, 70%, 75%, 80%, 85%, 90% or more) less “off-target” gene silencing when compared to similar constructs without the specified 5′-end modification, thus greatly improving the overall specificity of the RNAi reagent or therapeutics.

As used herein, “off-target” gene silencing refers to unintended gene silencing due to, for example, spurious sequence homology between the antisense (guide) sequence and the unintended target mRNA sequence.

According to this aspect of the disclosure, certain guide strand modifications further increase nuclease stability, and/or lower interferon induction, without significantly decreasing RNAi activity (or no decrease in RNAi activity at all).

Certain combinations of modifications may result in further unexpected advantages, as partly manifested by enhanced ability to inhibit target gene expression, enhanced serum stability, and/or increased target specificity, etc.

In certain embodiments, the guide strand comprises a 2′-O-methyl modified nucleotide at the 2^{nd} nucleotide on the 5′-end of the guide strand and no other modified nucleotides.

In other aspects, the chemically modified double stranded nucleic acid molecule structures of the present disclosure mediate sequence-dependent gene silencing by a microRNA mechanism. As used herein, the term “microRNA” (“miRNA”), also referred to in the art as “small temporal RNAs” (“stRNAs”), refers to a small (10-50 nucleotide) RNA which are genetically encoded (e.g., by viral, mammalian, or plant genomes) and are capable of directing or mediating RNA silencing. An “miRNA disorder” shall refer to a disease or disorder characterized by an aberrant expression or activity of an miRNA.

microRNAs are involved in down-regulating target genes in critical pathways, such as development and cancer, in mice, worms and mammals. Gene silencing through a microRNA mechanism is achieved by specific yet imperfect base-pairing of the miRNA and its target messenger RNA (mRNA). Various mechanisms may be used in microRNA-mediated down-regulation of target mRNA expression.

miRNAs are noncoding RNAs of approximately 22 nucleotides which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNAs are expressed by endogenous genes in vivo and are processed from a hairpin or stem-loop precursor (pre-miRNA or pri-miRNAs) by Dicer or other RNAses. miRNAs can exist transiently in vivo as a double stranded duplex but only one strand is taken up by the RISC complex to direct gene silencing.

In some embodiments, a version of chemically modified double stranded nucleic acid compounds, which are effective in cellular uptake and inhibition of miRNA activity, are described. Essentially, the compounds are similar to RISC entering versions, but large strand chemical modification patterns are made to block cleavage and act as an effective inhibitor of the RISC action. For example, the compound might be completely or mostly O-methyl modified with the phosphorothioate content described previously. For these types of compounds, the 5′ phosphorylation is not necessary in some embodiments. The presence of a double stranded region is preferred as it promotes cellular uptake and efficient RISC loading.

Another pathway that uses small RNAs as sequence-specific regulators is the RNA interference (RNAi) pathway, which is an evolutionarily conserved response to the presence of double stranded RNA (dsRNA) in the cell. The dsRNAs are cleaved into ˜20-base pair (bp) duplexes of small-interfering RNAs (siRNAs) by Dicer. These small RNAs get assembled into multiprotein effector complexes called RNA-induced silencing complexes (RISCs). The siRNAs then guide the cleavage of target mRNAs with perfect complementarity.

Some aspects of biogenesis, protein complexes, and function are shared between the siRNA pathway and the miRNA pathway. Single stranded polynucleotides may mimic the dsRNA in the siRNA mechanism, or the microRNA in the miRNA mechanism.

In certain embodiments, the modified RNAi constructs may have improved stability in serum and/or cerebral spinal fluid compared to an unmodified RNAi constructs having the same sequence.

In certain embodiments, the structure of the RNAi construct does not induce interferon response in primary cells, such as mammalian primary cells, including primary cells from human, mouse and other rodents, and other non-human mammals. In certain embodiments, the RNAi construct may also be used to inhibit expression of a target gene in an invertebrate organism.

To further increase the stability of the subject constructs in vivo, the 3′-end of the structure may be blocked by protective group(s). For example, protective groups such as inverted nucleotides, inverted abasic moieties, or amino-end modified nucleotides may be used. Inverted nucleotides may comprise an inverted deoxynucleotide. Inverted abasic moieties may comprise an inverted deoxyabasic moiety, such as a 3′,3′-linked or 5′,5′-linked deoxyabasic moiety.

The RNAi constructs of the disclosure are capable of inhibiting the synthesis of any target protein encoded by target gene(s). The disclosure includes methods to inhibit expression of a target gene either in a cell in vitro, or in vivo. As such, the RNAi constructs of the disclosure are useful for treating a patient with a disease characterized by the overexpression of a target gene.

The target gene can be endogenous or exogenous (e.g., introduced into a cell by a virus or using recombinant DNA technology) to a cell. Such methods may include introduction of RNA into a cell in an amount sufficient to inhibit expression of the target gene. By way of example, such an RNA molecule may have a guide strand that is complementary to the nucleotide sequence of the target gene, such that the composition inhibits expression of the target gene.

The disclosure also relates to vectors expressing the nucleic acids of the disclosure, and cells comprising such vectors or the nucleic acids. The cell may be a mammalian cell in vivo or in culture, such as a human cell.

The disclosure further relates to compositions comprising the subject RNAi constructs, and a pharmaceutically acceptable carrier or diluent.

The method may be carried out in vitro, ex vivo, or in vivo, in, for example, mammalian cells in culture, such as a human cell in culture.

The target cells (e.g., mammalian cell) may be contacted in the presence of a delivery reagent, such as a lipid (e.g., a cationic lipid) or a liposome.

Another aspect of the disclosure provides a method for inhibiting the expression of a target gene in a mammalian cell, comprising contacting the mammalian cell with a vector expressing the subject RNAi constructs.

In one aspect of the disclosure, a longer duplex polynucleotide is provided, including a first polynucleotide that ranges in size from about 16 to about 30 nucleotides; a second polynucleotide that ranges in size from about 26 to about 46 nucleotides, wherein the first polynucleotide (the antisense strand) is complementary to both the second polynucleotide (the sense strand) and a target gene, and wherein both polynucleotides form a duplex and wherein the first polynucleotide contains a single stranded region longer than 6 bases in length and is modified with alternative chemical modification pattern, and/or includes a conjugate moiety that facilitates cellular delivery. In this embodiment, between about 40% to about 90% of the nucleotides of the passenger strand between about 40% to about 90% of the nucleotides of the guide strand, and between about 40% to about 90% of the nucleotides of the single stranded region of the first polynucleotide are chemically modified nucleotides.

In an embodiment, the chemically modified nucleotide in the polynucleotide duplex may be any chemically modified nucleotide known in the art, such as those discussed in detail above. In a particular embodiment, the chemically modified nucleotide is selected from the group consisting of 2′ F modified nucleotides, 2′-O-methyl modified and 2′deoxy nucleotides. In another particular embodiment, the chemically modified nucleotides results from “hydrophobic modifications” of the nucleotide base. In another particular embodiment, the chemically modified nucleotides are phosphorothioates. In an additional particular embodiment, chemically modified nucleotides are combination of phosphorothioates, 2′-O-methyl, 2′deoxy, hydrophobic modifications and phosphorothioates. As these groups of modifications refer to modification of the ribose ring, back bone and nucleotide, it is feasible that some modified nucleotides will carry a combination of all three modification types.

In another embodiment, the chemical modification is not the same across the various regions of the duplex. In a particular embodiment, the first polynucleotide (the passenger strand), has a large number of diverse chemical modifications in various positions. For this polynucleotide up to 90% of nucleotides might be chemically modified and/or have mismatches introduced.

In another embodiment, chemical modifications of the first or second polynucleotide include, but not limited to, 5′ position modification of Uridine and Cytosine (4-pyridyl, 2-pyridyl, indolyl, phenyl (C₆H₅OH); tryptophanyl (C₈H₆N)CH₂CH(NH₂) CO), isobutyl, butyl, aminobenzyl; phenyl; naphthyl, etc.), where the chemical modification might alter base pairing capabilities of a nucleotide. For the guide strand an important feature of this aspect of the disclosure is the position of the chemical modification relative to the 5′ end of the antisense and sequence. For example, chemical phosphorylation of the 5′ end of the guide strand is usually beneficial for efficacy. O-methyl modifications in the seed region of the sense strand (position 2-7 relative to the 5′ end) are not generally well tolerated, whereas 2′F and deoxy are well tolerated. The mid part of the guide strand and the 3′ end of the guide strand are more permissive in a type of chemical modifications applied. Deoxy modifications are not tolerated at the 3′ end of the guide strand.

A unique feature of this aspect of the disclosure involves the use of hydrophobic modification on the bases. In one embodiment, the hydrophobic modifications are preferably positioned near the 5′ end of the guide strand, in other embodiments, they localized in the middle of the guides strand, in other embodiment they localized at the 3′ end of the guide strand and yet in another embodiment they are distributed thought the whole length of the polynucleotide. The same type of patterns is applicable to the passenger strand of the duplex.

The other part of the molecule is a single stranded region. The single stranded region is expected to range from 7 to 40 nucleotides.

In one embodiment, the single stranded region of the first polynucleotide contains modifications selected from the group consisting of between 40% and 90% hydrophobic base modifications, between 40%-90% phosphorothioates, between 40%-90% modification of the ribose moiety, and any combination of the preceding.

Efficiency of guide strand (first polynucleotide) loading into the RISC complex might be altered for heavily modified polynucleotides, so in one embodiment, the duplex polynucleotide includes a mismatch between nucleotide 9, 11, 12, 13, or 14 on the guide strand (first polynucleotide) and the opposite nucleotide on the sense strand (second polynucleotide) to promote efficient guide strand loading.

### Duplex Characteristics

Double stranded oligonucleotides of the disclosure may be formed by two separate complementary nucleic acid strands. Duplex formation can occur either inside or outside the cell containing the target gene.

As used herein, the term “duplex” includes the region of the double stranded nucleic acid molecule(s) that is (are) hydrogen bonded to a complementary sequence. Double stranded oligonucleotides of the disclosure may comprise a nucleotide sequence that is sense to a target gene and a complementary sequence that is antisense to the target gene. The sense and antisense nucleotide sequences correspond to the target gene sequence, e.g., are identical or are sufficiently identical to effect target gene inhibition (e.g., are about at least about 98% identical, 96% identical, 94%, 90% identical, 85% identical, or 80% identical) to the target gene sequence.

In certain embodiments, the double stranded oligonucleotide of the disclosure is double stranded over its entire length, i.e., with no overhanging single stranded sequence at either end of the molecule, i.e., is blunt-ended. In other embodiments, the individual nucleic acid molecules can be of different lengths. In other words, a double stranded oligonucleotide of the disclosure is not double stranded over its entire length. For instance, when two separate nucleic acid molecules are used, one of the molecules, e.g., the first molecule comprising an antisense sequence, can be longer than the second molecule hybridizing thereto (leaving a portion of the molecule single stranded). Likewise, when a single nucleic acid molecule is used a portion of the molecule at either end can remain single stranded.

In one embodiment, a double stranded oligonucleotide of the disclosure contains mismatches and/or loops or bulges, but is double stranded over at least about 70% of the length of the oligonucleotide. In another embodiment, a double stranded oligonucleotide of the disclosure is double stranded over at least about 80% of the length of the oligonucleotide. In another embodiment, a double stranded oligonucleotide of the disclosure is double stranded over at least about 90%-95% of the length of the oligonucleotide. In another embodiment, a double stranded oligonucleotide of the disclosure is double stranded over at least about 96%-98% of the length of the oligonucleotide. In certain embodiments, the double stranded oligonucleotide of the disclosure contains at least or up to 1, 2, 3, 4, 5, 6, 7, 8, 9, 10, 11, 12, 13, 14, or 15 mismatches.

### Modifications

The nucleotides of the disclosure may be modified at various locations, including the sugar moiety, the phosphodiester linkage, and/or the base.

In some embodiments, the base moiety of a nucleoside may be modified. For example, a pyrimidine base may be modified at the 2, 3, 4, 5, and/or 6 position of the pyrimidine ring. In some embodiments, the exocyclic amine of cytosine may be modified. A purine base may also be modified. For example, a purine base may be modified at the 1, 2, 3, 6, 7, or 8 position. In some embodiments, the exocyclic amine of adenine may be modified. In some cases, a nitrogen atom in a ring of a base moiety may be substituted with another atom, such as carbon. A modification to a base moiety may be any suitable modification. Examples of modifications are known to those of ordinary skill in the art. In some embodiments, the base modifications include alkylated purines or pyrimidines, acylated purines or pyrimidines, or other heterocycles.

In some embodiments, a pyrimidine may be modified at the 5 position. For example, the 5 position of a pyrimidine may be modified with an alkyl group, an alkynyl group, an alkenyl group, an acyl group, or substituted derivatives thereof. In other examples, the 5 position of a pyrimidine may be modified with a hydroxyl group or an alkoxyl group or substituted derivative thereof. Also, the N⁴ position of a pyrimidine may be alkylated. In still further examples, the pyrimidine 5-6 bond may be saturated, a nitrogen atom within the pyrimidine ring may be substituted with a carbon atom, and/or the O² and O⁴ atoms may be substituted with sulfur atoms. It should be understood that other modifications are possible as well.

In other examples, the N⁷ position and/or N² and/or N³ position of a purine may be modified with an alkyl group or substituted derivative thereof. In further examples, a third ring may be fused to the purine bicyclic ring system and/or a nitrogen atom within the purine ring system may be substituted with a carbon atom. It should be understood that other modifications are possible as well.

Non-limiting examples of pyrimidines modified at the 5 position are disclosed in U.S. Pat. Nos. 5,591,843, 7,205,297, 6,432,963, and 6,020,483; non-limiting examples of pyrimidines modified at the N⁴ position are disclosed in U.S. Pat. No. 5,580,731; non-limiting examples of purines modified at the 8 position are disclosed in U.S. Pat. Nos. 6,355,787 and 5,580,972; non-limiting examples of purines modified at the N⁶ position are disclosed in U.S. Pat. Nos. 4,853,386, 5,789,416, and 7,041,824; and non-limiting examples of purines modified at the 2 position are disclosed in U.S. Pat. Nos. 4,201,860 and 5,587,469, all of which are incorporated herein by reference.

Non-limiting examples of modified bases include N⁴, N⁴-ethanocytosine, 7-deazaxanthosine, 7-deazaguanosine, 8-oxo-N°-methyladenine, 4-acetylcytosine, 5-(carboxyhydroxylmethyl) uracil, 5-fluorouracil, 5-bromouracil, 5-carboxymethylaminomethyl-2-thiouracil, 5-carboxymethylaminomethyl uracil, dihydrouracil, inosine, N°-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy aminomethyl-2-thiouracil, 5-methoxyuracil, 2-methylthio-N⁶-isopentenyladenine, pseudouracil, 5-methyl-2-thiouracil, 2-thiouracil, 4-thiouracil, 5-methyluracil, 2-thiocytosine, and 2,6-diaminopurine. In some embodiments, the base moiety may be a heterocyclic base other than a purine or pyrimidine. The heterocyclic base may be optionally modified and/or substituted.

Sugar moieties include natural, unmodified sugars, e.g., monosaccharide (such as pentose, e.g., ribose, deoxyribose), modified sugars and sugar analogs. In general, possible modifications of nucleomonomers, particularly of a sugar moiety, include, for example, replacement of one or more of the hydroxyl groups with a halogen, a heteroatom, an aliphatic group, or the functionalization of the hydroxyl group as an ether, an amine, a thiol, or the like.

One useful group of modified nucleomonomers is 2′-O-methyl nucleotides. Such 2′-O-methyl nucleotides may be referred to as “methylated,” and the corresponding nucleotides may be made from unmethylated nucleotides followed by alkylation or directly from methylated nucleotide reagents. Modified nucleomonomers may be used in combination with unmodified nucleomonomers. For example, an oligonucleotide of the disclosure may contain both methylated and unmethylated nucleomonomers.

Some exemplary modified nucleomonomers include sugar- or backbone-modified ribonucleotides. Modified ribonucleotides may contain a non-naturally occurring base (instead of a naturally occurring base), such as uridines or cytidines modified at the 5′-position, e.g., 5′-(2-amino) propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides may have the 2′-OH group replaced by a H, alxoxy (or OR), R or alkyl, halogen, SH, SR, amino (such as NH₂, NHR, NR₂.), or CN group, wherein R is lower alkyl, alkenyl, or alkynyl.

Modified ribonucleotides may also have the phosphodiester group connecting to adjacent ribonucleotides replaced by a modified group, e.g., of phosphorothioate group. More generally, the various nucleotide modifications may be combined.

Although the antisense (guide) strand may be substantially identical to at least a portion of the target gene (or genes), at least with respect to the base pairing properties, the sequence need not be perfectly identical to be useful, e.g., to inhibit expression of a target gene's phenotype. Generally, higher homology can be used to compensate for the use of a shorter antisense gene. In some cases, the antisense strand generally will be substantially identical (although in antisense orientation) to the target gene.

The use of 2′-O-methyl modified RNA may also be beneficial in circumstances in which it is desirable to minimize cellular stress responses. RNA having 2′-O-methyl nucleomonomers may not be recognized by cellular machinery that is thought to recognize unmodified RNA. The use of 2′-O-methylated or partially 2′-O-methylated RNA may avoid the interferon response to double stranded nucleic acids, while maintaining target RNA inhibition. This may be useful, for example, for avoiding the interferon or other cellular stress responses, both in short RNAi (e.g., siRNA) sequences that induce the interferon response, and in longer RNAi sequences that may induce the interferon response.

Overall, modified sugars may include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described (Augustyns, K., et al., Nucl. Acids. Res. 18:4711 (1992)). Exemplary nucleomonomers can be found, e.g., in U.S. Pat. No. 5,849,902, incorporated by reference herein.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present disclosure may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure. Isomeric mixtures containing any of a variety of isomer ratios may be utilized in accordance with the present disclosure. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present disclosure. Those of ordinary skill in the art will readily appreciate that analogous ratios are contemplated for more complex isomer mixtures.

If, for instance, a particular enantiomer of a compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

In certain embodiments, oligonucleotides of the disclosure comprise 3′ and 5′ termini (except for circular oligonucleotides). In one embodiment, the 3′ and 5′ termini of an oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′-+3′ internucleotide linkage. For example, the alkyloxy radical can be methoxy, ethoxy, or isopropoxy, and preferably, ethoxy. Optionally, the 3′->3′linked nucleotide at the 3′ terminus can be linked by a substitute linkage. To reduce nuclease degradation, the 5′ most 3′-+5′ linkage can be a modified linkage, e.g., a phosphorothioate or a P-alkyloxyphosphotriester linkage. Preferably, the two 5′ most 3′->5′ linkages are modified linkages. Optionally, the 5′ terminal hydroxy moiety can be esterified with a phosphorus containing moiety, e.g., phosphate, phosphorothioate, or P-ethoxyphosphate.

One of ordinary skill in the art will appreciate that the synthetic methods, as described herein, utilize a variety of protecting groups. By the term “protecting group,” as used herein, it is meant that a particular functional moiety, e.g., O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p.p′-dinitrobenzhydryl, 5-dibenzosuberyl, triphenylmethyl, a-naphthyldiphenylmethyl, p-methoxyphenyldiphenylmethyl, di (p-methoxyphenyl)phenylmethyl, tri (p-methoxyphenyl)methyl, 4-(4′-bromophenacyloxyphenyl)diphenylmethyl, 4,4′,4″-tris (4,5-dichlorophthalimidophenyl)methyl, 4,4′,4″-tris (levulinoyloxyphenyl)methyl, 4,4′,4″-tris (benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, 1-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o)-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-1-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10, 10, 10, 10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-1-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (1-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o)-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di (2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o)-nitrophenoxyacetamide, acetoacetamide, (N′-dithiobenzyloxycarbonylamino) acetamide, 3-(p-hydroxyphenyl) propanamide, 3-(o-nitrophenyl) propanamide, 2-methyl-2-(0)-nitrophenoxy) propanamide, 2-methyl-2-(o)-phenylazophenoxy) propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di (4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl (pentacarbonylchromium- or tungsten) carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6, —trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein. However, it will be appreciated that the present disclosure is not intended to be limited to these protecting groups; rather, a variety of additional equivalent protecting groups can be readily identified using the above criteria and utilized in the method of the present disclosure. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this disclosure, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Furthermore, this disclosure is not intended to be limited in any manner by the permissible substituents of organic compounds. Combinations of substituents and variables envisioned by this disclosure are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “lower alkyl” is used to indicate those alkyl groups (cyclic, acyclic, substituted, unsubstituted, branched, or unbranched) having 1-6 carbon atoms.

In certain embodiments, the alkyl, alkenyl, and alkynyl groups employed in the disclosure contain 1-20 aliphatic carbon atoms. In certain other embodiments, the alkyl, alkenyl, and alkynyl groups employed in the disclosure contain 1-10 aliphatic carbon atoms. In yet other embodiments, the alkyl, alkenyl, and alkynyl groups employed in the disclosure contain 1-8 aliphatic carbon atoms. In still other embodiments, the alkyl, alkenyl, and alkynyl groups employed in the disclosure contain 1-6 aliphatic carbon atoms. In yet other embodiments, the alkyl, alkenyl, and alkynyl groups employed in the disclosure contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

Some examples of substituents of the above-described aliphatic (and other) moieties of compounds of the disclosure include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON (Rₓ)₂; —OC(O)Rₓ; —OCO₂Rₓ; —OCON (Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

The terms “halo” and “halogen” as used herein refer to an atom selected from fluorine, chlorine, bromine, and iodine.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, unless otherwise specified, the term alkynyl includes both “unsubstituted alkynyls” and “substituted alkynyls,” the latter of which refers to alkynyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with independently selected groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulffiydryl, alkylthio, arylthio, thiocarboxylate, sulfates, alkylsulfmyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “heteroatom” includes atoms of any element other than carbon or hydrogen. Preferred heteroatoms are nitrogen, oxygen, sulfur and phosphorus.

The term “hydroxy” or “hydroxyl” includes groups with an —OH or —O″ (with an appropriate counterion).

The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C (halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃₀ (CR′R″)₀₋₃H, (CR′R″)₀₋₃S(O)₀₋₂R′, (CR′R″)₀₋₃₀ (CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or α-(CH₂)₂O (CH₂)₂-group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The terms “polynucleotide,” “nucleotide sequence,” “nucleic acid,” “nucleic acid molecule,” “nucleic acid sequence,” and “oligonucleotide” refer to a polymer of two or more nucleotides. The polynucleotides can be DNA, RNA, or derivatives or modified versions thereof. The polynucleotide may be single stranded or double stranded. The polynucleotide can be modified at the base moiety, sugar moiety, or phosphate backbone, for example, to improve stability of the molecule, its hybridization parameters, etc. The polynucleotide may comprise a modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. The olynucleotide may comprise a modified sugar moiety (e.g., 2′-fluororibose, ribose, 2′-deoxyribose, 2′-O-methylcytidine, arabinose, and hexose), and/or a modified phosphate moiety (e.g., phosphorothioates and 5′-N-phosphoramidite linkages). A nucleotide sequence typically carries genetic information, including the information used by cellular machinery to make proteins and enzymes. These terms include double or single stranded genomic and cDNA, RNA, any synthetic and genetically manipulated polynucleotide, and both sense and antisense polynucleotides. This includes single and double stranded molecules, i.e., DNA-DNA, DNA-RNA, and RNA-RNA hybrids, as well as “protein nucleic acids” (PNA) formed by conjugating bases to an amino acid backbone.

The term “targeting moiety” or “targeting ligand” includes, but is not limited to, N-acetylglucosamine acetyl or N-acetyl galactosamine (GalNac).

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N6-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl) uracil, 5-(1-propynyl) cytosine and 4,4-ethanocytosine). Other examples of suitable bases include non-purinyl and non-pyrimidinyl bases such as 2-aminopyridine and triazines.

In a preferred embodiment, the nucleomonomers of an oligonucleotide of the disclosure are RNA nucleotides. In another preferred embodiment, the nucleomonomers of an oligonucleotide of the disclosure are modified RNA nucleotides. Thus, the oligonucleotides contain modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2nd Ed., Wiley-Interscience, New York, 1999).

The term “nucleotide” includes nucleosides which further comprise a phosphate group or a phosphate analog.

The nucleic acid molecules may be associated with a hydrophobic moiety for targeting and/or delivery of the molecule to a cell. In certain embodiments, the hydrophobic moiety is associated with the nucleic acid molecule through a linker. In certain embodiments, the association is through non-covalent interactions. In other embodiments, the association is through a covalent bond. The nucleic acid molecules may be associated with a targeting ligand moiety for targeting and/or delivery of the molecule to a cell. In certain embodiments, the targeting ligand moiety is associated with the nucleic acid molecule as a phosphoroamidite or alternatively through a linker. In certain embodiments, the association is through non-covalent interactions. In other embodiments, the association is through a covalent bond. Any linker known in the art may be used to associate the nucleic acid with the hydrophobic moiety. Linkers known in the art are described in published international PCT applications, WO 92/03464, WO 95/23162, WO 2008/021157, WO 2009/021157, WO 2009/134487, WO 2009/126933, U.S. Patent Application Publication 2005/0107325, U.S. Pat. No. 5,414,077, U.S.

U.S. Pat. Nos. 5,419,966, 5,512,667, U.S. Patent 5,646, 126, and U.S. Pat. No. 5,652,359, which are incorporated herein by reference. The linker may be as simple as a covalent bond to a multi-atom linker. The linker may be cyclic or acyclic. The linker may be optionally substituted. In certain embodiments, the linker is capable of being cleaved from the nucleic acid. In certain embodiments, the linker is capable of being hydrolyzed under physiological conditions. In certain embodiments, the linker is capable of being cleaved by an enzyme (e.g., an esterase or phosphodiesterase). In certain embodiments, the linker comprises a spacer element to separate the nucleic acid from the hydrophobic moiety. The spacer element may include one to thirty carbon or heteroatoms. In certain embodiments, the linker and/or spacer element comprises protonatable functional groups. Such protonatable functional groups may promote the endosomal escape of the nucleic acid molecule. The protonatable functional groups may also aid in the delivery of the nucleic acid to a cell, for example, neutralizing the overall charge of the molecule. In other embodiments, the linker and/or spacer element is biologically inert (that is, it does not impart biological activity or function to the resulting nucleic acid molecule).

In certain embodiments, the nucleic acid molecule with a targeting moiety and/or a linker and hydrophobic moiety is of the formulae described herein. In certain embodiments, the nucleic acid molecule is of the formula:

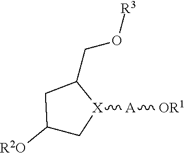

wherein
X is N or CH;
A is a bond; substituted or unsubstituted, cyclic or acyclic, branched or unbranched aliphatic; or substituted or unsubstituted, cyclic or acyclic, branched or unbranched heteroaliphatic;
R¹ is a hydrophobic moiety;
R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and
R³ is a nucleic acid.

In certain embodiments, the molecule is of the formula:

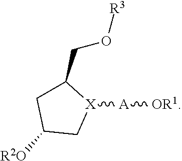

In certain embodiments, the molecule is of the formula:

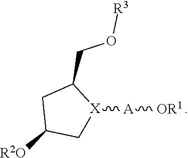

In certain embodiments, the molecule is of the formula:

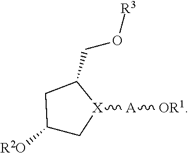

In certain embodiments, the molecule is of the formula:

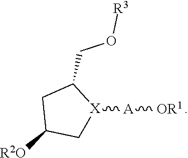

In certain embodiments, X is N. In certain embodiments, X is CH.

In certain embodiments, A is a bond. In certain embodiments, A is substituted or unsubstituted, cyclic or acyclic, branched or unbranched aliphatic. In certain embodiments, A is acyclic, substituted or unsubstituted, branched or unbranched aliphatic. In certain embodiments, A is acyclic, substituted, branched or unbranched aliphatic. In certain embodiments, A is acyclic, substituted, unbranched aliphatic. In certain embodiments, A is acyclic, substituted, unbranched alkyl. In certain embodiments, A is acyclic, substituted, unbranched C₁₋₂₀ alkyl. In certain embodiments, A is acyclic, substituted, unbranched C₁₋₁₂ alkyl. In certain embodiments, A is acyclic, substituted, unbranched C₁₋₁₀ alkyl. In certain embodiments, A is acyclic, substituted, unbranched C₁₋₈ alkyl. In certain embodiments, A is acyclic, substituted, unbranched C₁₋₆ alkyl. In certain embodiments, A is substituted or unsubstituted, cyclic or acyclic, branched or unbranched heteroaliphatic. In certain embodiments, A is acyclic, substituted or unsubstituted, branched or unbranched heteroaliphatic. In certain embodiments, A is acyclic, substituted, branched or unbranched heteroaliphatic. In certain embodiments, A is acyclic, substituted, unbranched heteroaliphatic.

In certain embodiments, A is of the formula:

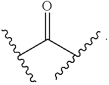 [SMILES: *C(*)=O]

In certain embodiments, A is of one of the formulae:

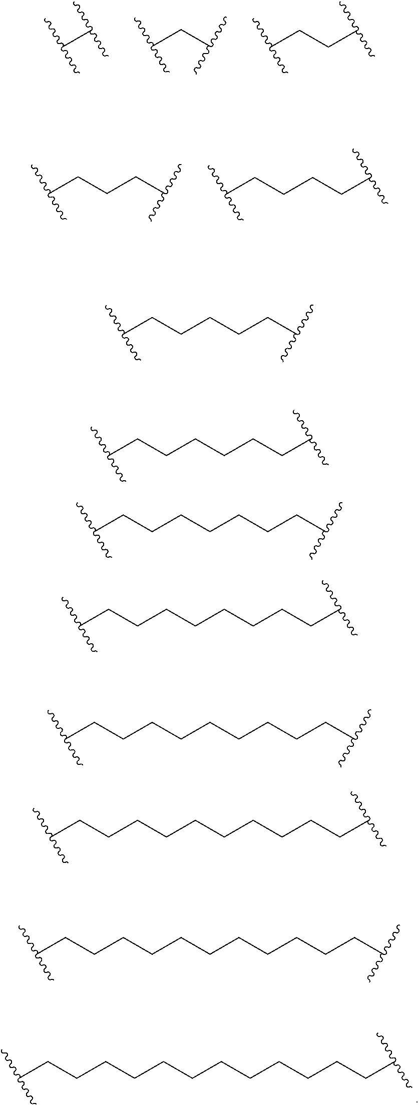

In certain embodiments, A is of one of the formulae:

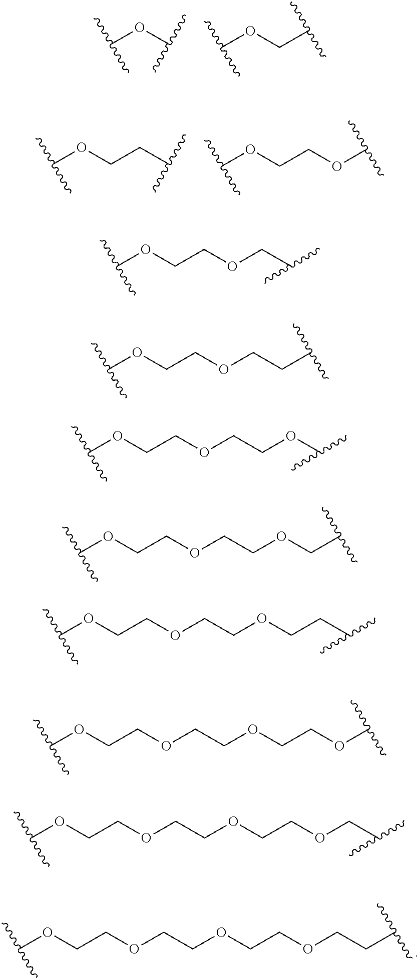

In certain embodiments, A is of one of the formulae:

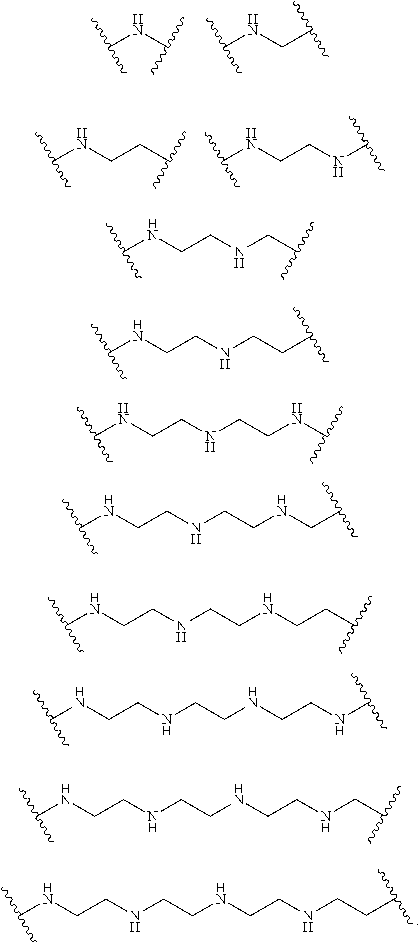

In certain embodiments, A is of the formula:

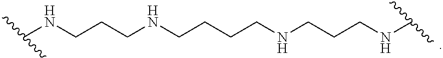 [SMILES: *NCCCNCCCCNCCCN*]

In certain embodiments, A is of the formula:

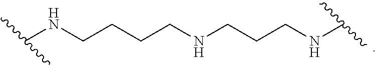 [SMILES: *NCCCCNCCCN*]

In certain embodiments, A is of the formula:

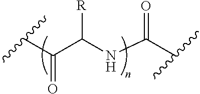

wherein each occurrence of R is independently the side chain of a natural or unnatural amino acid; and n is an integer between 1 and 20, inclusive. In certain embodiments, A is of the formula:

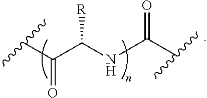

In certain embodiments, each occurrence of R is independently the side chain of a natural amino acid. In certain embodiments, n is an integer between 1 and 15, inclusive. In certain embodiments, n is an integer between 1 and 10, inclusive. In certain embodiments, n is an integer between 1 and 5, inclusive.

In certain embodiments, A is of the formula:

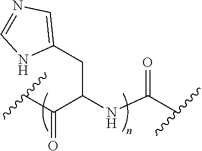

wherein n is an integer between 1 and 20, inclusive. In certain embodiments, A is of the formula:

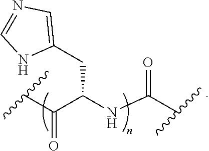

In certain embodiments, n is an integer between 1 and 15, inclusive. In certain embodiments, n is an integer between 1 and 10, inclusive. In certain embodiments, n is an integer between 1 and 5, inclusive.

In certain embodiments, A is of the formula:

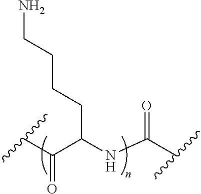

wherein n is an integer between 1 and 20, inclusive. In certain embodiments, A is of the formula:

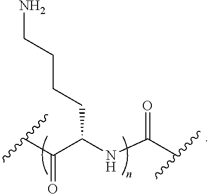

In certain embodiments, n is an integer between 1 and 15, inclusive. In certain embodiments, n is an integer between 1 and 10, inclusive. In certain embodiments, n is an integer between 1 and 5, inclusive.

In certain embodiments, the molecule is of the formula:

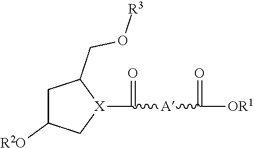

wherein X, R¹, R², and R³ are as defined herein; and
A′ is substituted or unsubstituted, cyclic or acyclic, branched or unbranched aliphatic; or substituted or unsubstituted, cyclic or acyclic, branched or unbranched heteroaliphatic.

In certain embodiments, A′ is of one of the formulae:

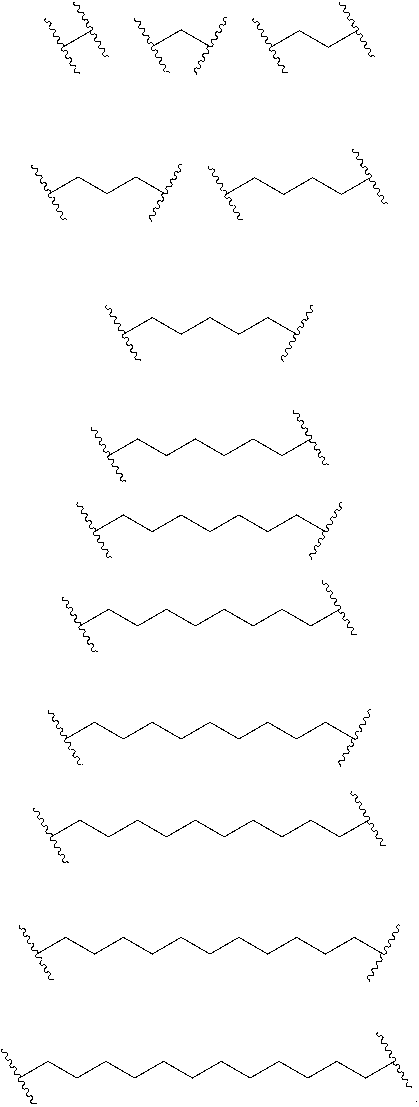

In certain embodiments, A is of one of the formulae:

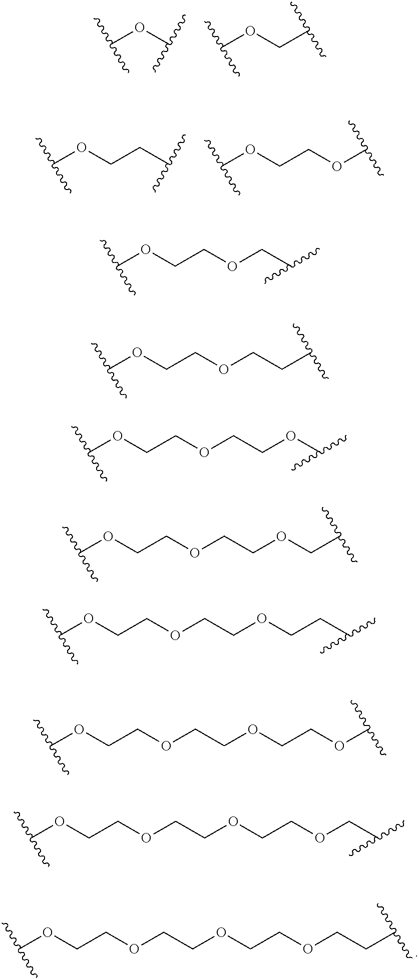

In certain embodiments, A is of one of the formulae:

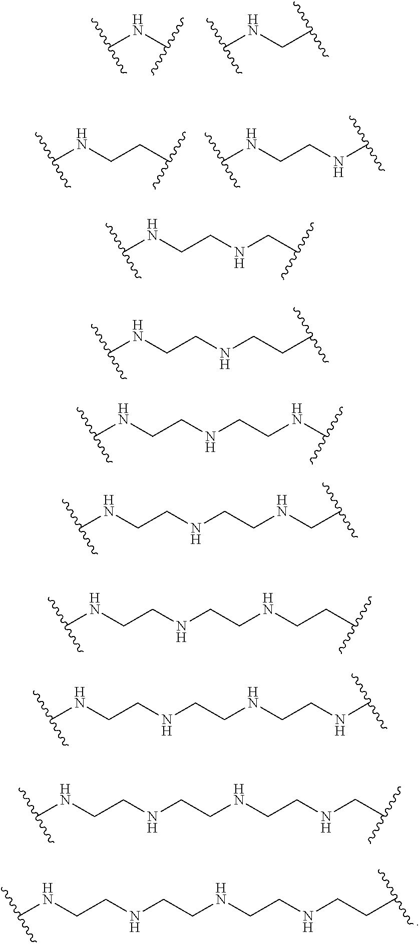

In certain embodiments. A is of the formula:

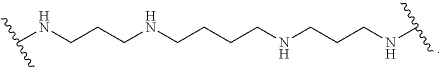 [SMILES: *NCCCNCCCCNCCCN*]

In certain embodiments, A is of the formula:

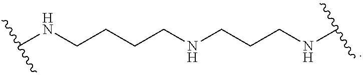 [SMILES: *NCCCCNCCCN*]

In certain embodiments, R¹ is a steroid. In certain embodiments, R¹ is a cholesterol. In certain embodiments, R¹ is a lipophilic vitamin. In certain embodiments, R¹ is a vitamin A. In certain embodiments, R¹ is a vitamin E.

In certain embodiments, R¹ is of the formula:

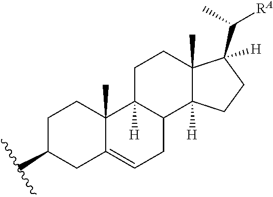

wherein R^{A} is substituted or unsubstituted, cyclic or acyclic, branched or unbranched aliphatic; or substituted or unsubstituted, cyclic or acyclic, branched or unbranched heteroaliphatic.

In certain embodiments, R¹ is of the formula:

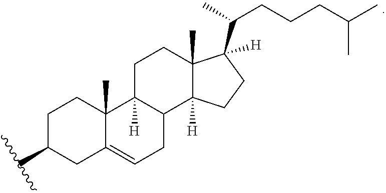

In certain embodiments, R¹ is of the formula:

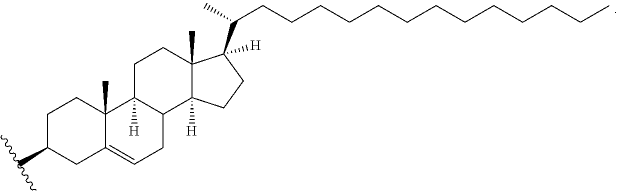

In certain embodiments, R¹ is of the formula:

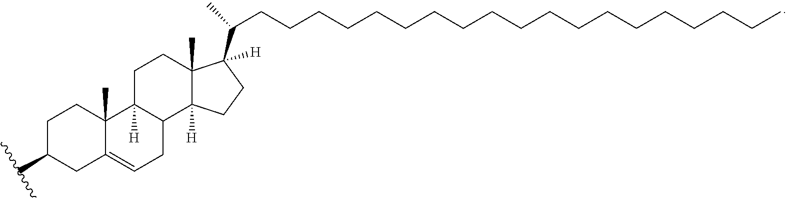

In certain embodiments, R¹ is of the formula:

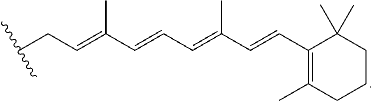 [SMILES: *C/C=C(C)/C=C/C=C(C)/C=C/C1=C(C)CCCC1(C)C]

In certain embodiments, R¹ is of the formula:

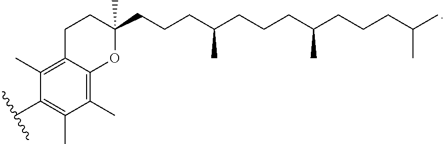 [SMILES: *c1c(C)c(C)c2c(c1C)CC[C@@](C)(CCC[C@H](C)CCC[C@H](C)CCCC(C)C)O2]

In certain embodiments, the nucleic acid molecule is of the formula:

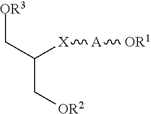

wherein
X is N or CH;
A is a bond; substituted or unsubstituted, cyclic or acyclic, branched or unbranched aliphatic; or substituted or unsubstituted, cyclic or acyclic, branched or unbranched heteroaliphatic;
R¹ is a hydrophobic moiety;
R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and
R³ is a nucleic acid.

In certain embodiments, the nucleic acid molecule is of the formula:

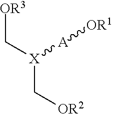

wherein
X is N or CH;
A is a bond; substituted or unsubstituted, cyclic or acyclic, branched or unbranched aliphatic; or substituted or unsubstituted, cyclic or acyclic, branched or unbranched heteroaliphatic;
R¹ is a hydrophobic moiety;
R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and
R³ is a nucleic acid.

In certain embodiments, the nucleic acid molecule is of the formula:

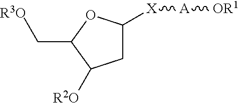

wherein
X is N or CH;
A is a bond; substituted or unsubstituted, cyclic or acyclic, branched or unbranched aliphatic; or substituted or unsubstituted, cyclic or acyclic, branched or unbranched heteroaliphatic;
R¹ is a hydrophobic moiety;
R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and
R³ is a nucleic acid. In certain embodiments, the nucleic acid molecule is of the formula:

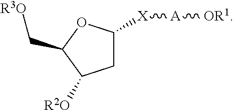

In certain embodiments, the nucleic acid molecule is of the formula:

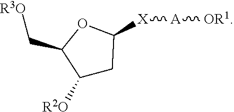

In certain embodiments, the nucleic acid molecule is of the formula:

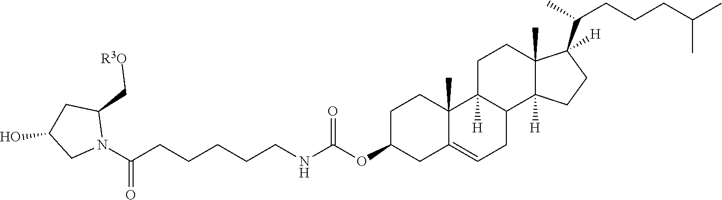

wherein R³ is a nucleic acid.

In certain embodiments, the nucleic acid molecule is of the formula:

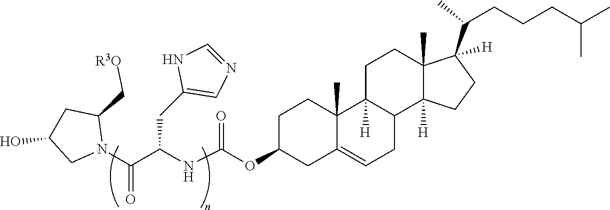

wherein R³ is a nucleic acid; and
n is an integer between 1 and 20, inclusive.

In certain embodiments, the nucleic acid molecule is of the formula:

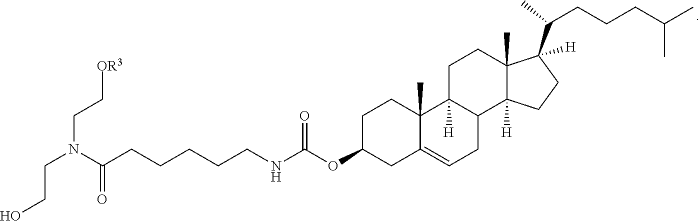

In certain embodiments, the nucleic acid molecule is of the formula:

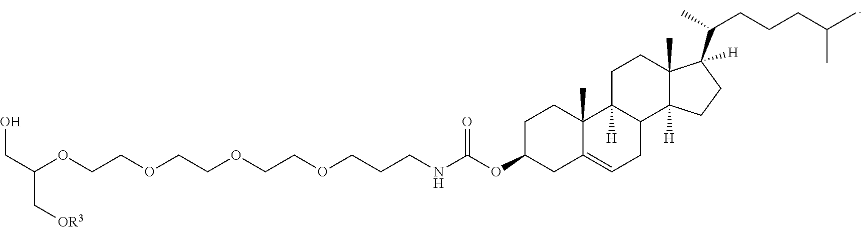

In certain embodiments, the nucleic acid molecule is of the formula:

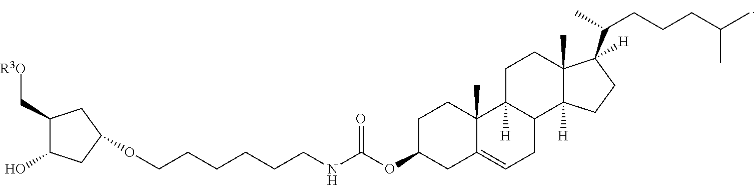

In certain embodiments, the nucleic acid molecule is of the formula:

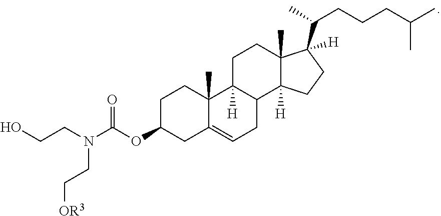

In certain embodiments, the nucleic acid molecule is of the formula:

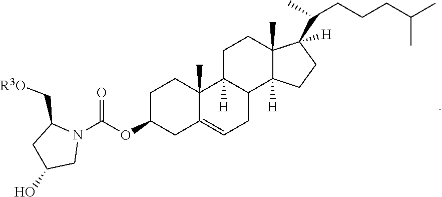

As used herein, the term “linkage” includes a naturally occurring, unmodified phosphodiester moiety (—O—(PO²—)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothioate linkages.

In certain embodiments, oligonucleotides of the disclosure comprise hydrophobically modified nucleotides or “hydrophobic modifications.” As used herein “hydrophobic modifications” refers to bases that are modified such that (1) overall hydrophobicity of the base is significantly increased, and/or (2) the base is still capable of forming close to regular Watson-Crick interaction. Several non-limiting examples of base modifications include 5-position uridine and cytidine modifications such as phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; and naphthyl.

Other types of conjugates that can be attached to the end (3′ or 5′ end), a loop region, or any other parts of a chemically modified double stranded nucleic acid molecule include a sterol, sterol type molecule, peptide, small molecule, protein, etc. In some embodiments, a chemically modified double stranded nucleic acid molecule, such as an sd-rxRNA (INTASYL™), may contain more than one conjugate (same or different chemical nature). In some embodiments, the conjugate is cholesterol. Alternatively, in some embodiments, the conjugate is GalNac

In some embodiments, the first nucleotide relative to the 5′end of the guide strand has a 2′-O-methyl modification, optionally wherein the 2′-O-methyl modification is a 5P-2′O-methyl U modification, or a 5′ vinyl phosphonate 2′-O-methyl U modification. Another way to increase target gene specificity, or to reduce off-target silencing effect, is to introduce a 2′-modification (such as the 2′-O methyl modification) at a position corresponding to the second 5′-end nucleotide of the guide sequence. Antisense (guide) sequences of the disclosure can be “chimeric oligonucleotides” which comprise an RNA-like and a DNA-like region.

The language “RNase H activating region” includes a region of an oligonucleotide, e.g., a chimeric oligonucleotide that is capable of recruiting RNase H to cleave the target RNA strand to which the oligonucleotide binds. Typically, the RNase activating region contains a minimal core (of at least about 3-5, typically between about 3-12, more typically, between about 5-12, and more preferably between about 5-10 contiguous nucleomonomers) of DNA or DNA-like nucleomonomers. (See, e.g., U.S. Pat. No. 5,849,902). Preferably, the RNase H activating region comprises about nine contiguous deoxyribose containing nucleomonomers.

The language “non-activating region” includes a region of an antisense sequence, e.g., a chimeric oligonucleotide that does not recruit or activate RNase H. Preferably, a non-activating region does not comprise phosphorothioate DNA. The oligonucleotides of the disclosure comprise at least one non-activating region. In one embodiment, the non-activating region can be stabilized against nucleases or can provide specificity for the target by being complementary to the target and forming hydrogen bonds with the target nucleic acid molecule, which is to be bound by the oligonucleotide.

In one embodiment, at least a portion of the contiguous polynucleotides are linked by a substitute linkage, e.g., a phosphorothioate linkage.

In certain embodiments, most or all of the nucleotides beyond the guide sequence (2′-modified or not) are linked by phosphorothioate linkages. Such constructs tend to have improved pharmacokinetics due to their higher affinity for serum proteins. The phosphorothioate linkages in the non-guide sequence portion of the polynucleotide generally do not interfere with guide strand activity, once the latter is loaded into RISC. In some embodiments, high levels of phosphorothioate modification can lead to improved delivery. In some embodiments, the guide and/or passenger strand is completely phosphorothioated.

Antisense (guide) sequences of the present disclosure may include “morpholino oligonucleotides.” Morpholino oligonucleotides are non-ionic and function by an RNase H-independent mechanism. Each of the 4 genetic bases (Adenine, Cytosine, Guanine, and Thymine/Uracil) of the morpholino oligonucleotides is linked to a 6-membered morpholine ring. Morpholino oligonucleotides are made by joining the 4 different subunit types by, e.g., non-ionic phosphorodiamidate inter-subunit linkages. Morpholino oligonucleotides have many advantages including: complete resistance to nucleases (Antisense & Nucl. Acid Drug Dev. 1996. 6:267); predictable targeting (Biochemica Biophysica Acta. 1999. 1489:141); reliable activity in cells (Antisense & Nucl. Acid Drug Dev. 1997. 7:63); excellent sequence specificity (Antisense & Nucl. Acid Drug Dev. 1997. 7:151); minimal non-antisense activity (Biochemica Biophysica Acta. 1999. 1489:141); and simple osmotic or scrape delivery (Antisense & Nucl. Acid Drug Dev. 1997. 7:291). Morpholino oligonucleotides are also preferred because of their non-toxicity at high doses. A discussion of the preparation of morpholino oligonucleotides can be found in Antisense & Nucl. Acid Drug Dev. 1997. 7:187.

The chemical modifications described herein are believed to promote single stranded polynucleotide loading into the RISC. Single stranded polynucleotides have been shown to be active in loading into RISC and inducing gene silencing. However, the level of activity for single stranded polynucleotides appears to be 2 to 4 orders of magnitude lower when compared to a duplex polynucleotide.

The present disclosure provides a description of the chemical modification patterns, which may (a) significantly increase stability of the single stranded polynucleotide (b) promote efficient loading of the polynucleotide into the RISC complex and (c) improve uptake of the single stranded nucleotide by the cell. The chemical modification patterns may include a combination of ribose, backbone, hydrophobic nucleoside and conjugate type of modifications. In addition, in some of the embodiments, the 5′ end of the single polynucleotide may be chemically phosphorylated.

In yet another embodiment, the present disclosure provides a description of the chemical modification patterns, which improve functionality of RISC inhibiting polynucleotides. Single stranded polynucleotides have been shown to inhibit activity of a preloaded RISC complex through the substrate competition mechanism. For these types of molecules, conventionally called antagomers, the activity usually requires high concentration and in vivo delivery is not very effective. The present disclosure provides a description of the chemical modification patterns, which may (a) significantly increase stability of the single stranded polynucleotide (b) promote efficient recognition of the polynucleotide by the RISC as a substrate and/or (c) improve uptake of the single stranded nucleotide by the cell. The chemical modification patterns may include a combination of ribose, backbone, hydrophobic nucleoside and conjugate type of modifications.

The modifications provided by the present disclosure are applicable to all polynucleotides. This includes single stranded RISC entering polynucleotides, single stranded RISC inhibiting polynucleotides, conventional duplexed polynucleotides of variable length (15-40 bp), asymmetric duplexed polynucleotides, and the like. Polynucleotides may be modified with wide variety of chemical modification patterns, including 5′ end, ribose, backbone and hydrophobic nucleoside modifications.

### Synthesis

Oligonucleotides of the disclosure can be synthesized by any method known in the art, e.g., using enzymatic synthesis and/or chemical synthesis. The oligonucleotides can be synthesized in vitro (e.g., using enzymatic synthesis and chemical synthesis) or in vivo (using recombinant DNA technology well known in the art).

In some embodiments, chemical synthesis is used for modified polynucleotides. Chemical synthesis of linear oligonucleotides is well known in the art and can be achieved by solution or solid phase techniques. Preferably, synthesis is by solid phase methods. Oligonucleotides can be made by any of several different synthetic procedures including the phosphoramidite, phosphite triester, H-phosphonate, and phosphotriester methods, typically by automated synthesis methods.

Oligonucleotide synthesis protocols are well known in the art and can be found, e.g., in U.S. Pat. No. 5,830,653; WO 98/13526; Stec et al. 1984. J. Am. Chem. Soc. 106:6077; Stec et al. 1985. J. Org. Chem. 50:3908; Stec et al. J. Chromatog. 1985. 326:263; LaPlanche et al. 1986. Nucl. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. Nos. 5,013,830; 5,214,135; 5,525,719; Kawasaki et al. 1993. J. Med. Chem. 36:831; WO 92/03568; U.S. Pat. Nos. 5,276,019; and 5,264,423.

The synthesis method selected can depend on the length of the desired oligonucleotide and such choice is within the skill of the ordinary artisan. For example, the phosphoramidite and phosphite triester method can produce oligonucleotides having 175 or more nucleotides, while the H-phosphonate method works well for oligonucleotides of less than 100 nucleotides. If modified bases are incorporated into the oligonucleotide, and particularly if modified phosphodiester linkages are used, then the synthetic procedures are altered as needed according to known procedures. In this regard, Uhlmann et al. (1990, Chemical Reviews 90:543-584) provide references and outline procedures for making oligonucleotides with modified bases and modified phosphodiester linkages. Other exemplary methods for making oligonucleotides are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984). Moreover, linear oligonucleotides of defined sequence, including some sequences with modified nucleotides, are readily available from several commercial sources.

The oligonucleotides may be purified by polyacrylamide gel electrophoresis, or by any of a number of chromatographic methods, including gel chromatography and high pressure liquid chromatography. To confirm a nucleotide sequence, especially unmodified nucleotide sequences, oligonucleotides may be subjected to DNA sequencing by any of the known procedures, including Maxam and Gilbert sequencing, Sanger sequencing, capillary electrophoresis sequencing, the wandering spot sequencing procedure, or by using selective chemical degradation of oligonucleotides bound to Hybond paper. Sequences of short oligonucleotides can also be analyzed by laser desorption mass spectroscopy or by fast atom bombardment (McNeal, et al., 1982, J. Am. Chem. Soc. 104:976; Viari, et al., 1987, Biomed. Environ. Mass Spectrom. 14:83; Grotjahn et al., 1982, Nuc. Acid Res. 10:4671). Sequencing methods are also available for RNA oligonucleotides.

The quality of oligonucleotides synthesized can be verified by testing the oligonucleotide by capillary electrophoresis and denaturing strong anion HPLC (SAX-HPLC) using, e.g., the method of Bergot and Egan. 1992. J. Chrom. 599:35.

Other exemplary synthesis techniques are well known in the art (see, e.g., Sambrook et al., Molecular Cloning: a Laboratory Manual, Second Edition (1989); DNA Cloning, Volumes I and II (DN Glover Ed. 1985); Oligonucleotide Synthesis (M J Gait Ed, 1984; Nucleic Acid Hybridisation (B D Hames and S J Higgins eds. 1984); A Practical Guide to Molecular Cloning (1984); or the series, Methods in Enzymology (Academic Press, Inc.)).

In certain embodiments, the subject RNAi constructs or at least portions thereof are transcribed from expression vectors encoding the subject constructs. Any art recognized vectors may be use for this purpose. The transcribed RNAi constructs may be isolated and purified, before desired modifications (such as replacing an unmodified sense strand with a modified one, etc.) are carried out.

### Delivery Carrier

Without wishing to be bound by any particular theory, the particular patterns of modifications on the passenger strand and guide strand of the double stranded nucleic acid molecules described herein (e.g., INTASYL™) may facilitate entry of the guide strand into the nucleus, where the guide strand mediates gene silencing (e.g., silencing of target genes, such as PD-1).

Without wishing to be bound by any theory, several potential mechanisms of action could account for this activity. For example, in some embodiments, the guide strand (e.g., antisense strand) of the nucleic acid molecule (e.g., INTASYL™) may dissociate from the passenger strand and enter into the nucleus as a single strand. Once in the nucleus the single stranded guide strand may associate with RNAse H or another ribonuclease and cleave the target (e.g., PD-1) (“Antisense mechanism of action”). In some embodiments, the guide strand (e.g., antisense strand) of the nucleic acid molecule (e.g., INTASYL™) may associate with an Argonaute (Ago) protein in the cytoplasm or outside the nucleus, forming a loaded Ago complex. This loaded Ago complex may translocate into the nucleus and then cleave the target (e.g., PD-1). In some embodiments, both strands (e.g. a duplex) of the nucleic acid molecule (e.g., INTASYL™) may enter the nucleus and the guide strand may associate with RNAse H, an Ago protein or another ribonuclease and cleaves the target (e.g., PD-1).

The skilled artisan appreciates that the sense strand of the double stranded molecules described herein (e.g., INTASYLTMsense strand) is not limited to delivery of a guide strand of the double stranded nucleic acid molecule described herein. Rather, in some embodiments, a passenger strand described herein is joined (e.g., covalently bound, non-covalently bound, conjugated, hybridized via a region of complementarity, etc.) to certain molecules (e.g., antisense oligonucleotides, ASO) for the purpose of targeting said other molecule to the nucleus of a cell. In some embodiments, the molecule joined to a sense strand described herein is a synthetic antisense oligonucleotide (ASO). In some embodiments, the sense strand joined to an anti-sense oligonucleotide is between 8-15 nucleotides long, chemically modified, and comprises a hydrophobic conjugate.

Without wishing to be bound by any particular theory, an ASO can be joined to a complementary passenger strand by hydrogen bonding. Accordingly, in some aspects, the disclosure provides a method of delivering a nucleic acid molecule to a cell, the method comprising administering an isolated nucleic acid molecule to a cell, wherein the isolated nucleic acid comprises a sense strand which is complementary to an anti-sense oligonucleotide (ASO), wherein the sense strand is between 8-15 nucleotides in length, comprises at least two phosphorothioate modifications, at least 50% of the pyrimidines in the sense strand are modified, and wherein the molecule comprises a hydrophobic conjugate.

### Uptake of Oligonucleotides by Cells

Oligonucleotides and oligonucleotide compositions are contacted with (i.e., brought into contact with, also referred to herein as administered or delivered to) and taken up by one or more cells or a cell lysate. The term “cells” includes prokaryotic and eukaryotic cells, preferably vertebrate cells, and, more preferably, mammalian cells. In some embodiments, the oligonucleotide compositions of the disclosure are contacted with bacterial cells. In some embodiments, the oligonucleotide compositions of the disclosure are contacted with eukaryotic cells (e.g., plant cell, mammalian cell, arthropod cell, such as insect cell). In some embodiments, the oligonucleotide compositions of the disclosure are contacted with stem cells. In some embodiments, the oligonucleotide compositions of the disclosure are contacted with immune cells, such as T cells (e.g., CD8+ T cells). In some embodiments, the T cells are CD8+ T cells or CD3+ T cells. In a preferred embodiment, the oligonucleotide compositions of the disclosure are contacted with human cells.

Oligonucleotide compositions of the disclosure can be contacted with cells in vitro, e.g., in a test tube or culture dish, (and may or may not be introduced into a subject) or in vivo, e.g., in a subject such as a mammalian subject, or ex vivo. In some embodiments, oligonucleotides are administered topically or through electroporation. Oligonucleotides are taken up by cells at a slow rate by endocytosis, but endocytosed oligonucleotides are generally sequestered and not available, e.g., for hybridization to a target nucleic acid molecule. In one embodiment, cellular uptake can be facilitated by electroporation or calcium phosphate precipitation. However, these procedures are only useful for in vitro or ex vivo embodiments, are not convenient and, in some cases, are associated with cell toxicity.

In another embodiment, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of oligonucleotides can also be mediated by the use of vectors (See e.g., Shi, Y. 2003. Trends Genet 2003 Jan. 19:9; Reichhart J M et al. Genesis. 2002. 34 (1-2): 1604, Yu et al. 2002. Proc. Natl. Acad Sci. USA 99:6047; Sui et al. 2002. Proc. Natl. Acad Sci. USA 99:5515) viruses, polyamine or polycation conjugates using compounds such as polylysine, protamine, or Ni, N12-bis (ethyl) spermine (see, e.g., Bartzatt, R. et al.1989. Biotechnol. Appl. Biochem. 11:133; Wagner E. et al. 1992. Proc. Natl. Acad. Sci. 88:4255).

In certain embodiments, the chemically modified double stranded nucleic acid molecules of the disclosure may be delivered by using various beta-glucan containing particles, referred to as GeRPs (glucan encapsulated RNA loaded particle), described in, and incorporated by reference from, U.S. Provisional Application No. 61/310,611, filed on Mar. 4, 2010 and entitled “Formulations and Methods for Targeted Delivery to Phagocyte Cells.” Such particles are also described in, and incorporated by reference from US Patent Publications US 2005/0281781 A1, and US 2010/0040656, and in PCT publications WO 2006/007372, and WO 2007/050643. The chemically modified double stranded nucleic acid molecule may be hydrophobically modified and optionally may be associated with a lipid and/or amphiphilic peptide. In certain embodiments, the beta-glucan particle is derived from yeast. In certain embodiments, the payload trapping molecule is a polymer, such as those with a molecular weight of at least about 1000 Da, 10,000 Da, 50,000 Da, 100 kDa, 500 kDa, etc. Preferred polymers include (without limitation) cationic polymers, chitosans, or PEI (polyethylenimine), etc.

Glucan particles can be derived from insoluble components of fungal cell walls such as yeast cell walls. In some embodiments, the yeast is Baker's yeast. Yeast-derived glucan molecules can include one or more of β-(1,3)-Glucan, β-(1,6)-Glucan, mannan and chitin. In some embodiments, a glucan particle comprises a hollow yeast cell wall whereby the particle maintains a three dimensional structure resembling a cell, within which it can complex with or encapsulate a molecule such as an RNA molecule. Some of the advantages associated with the use of yeast cell wall particles are availability of the components, their biodegradable nature, and their ability to be targeted to phagocytic cells.

In some embodiments, glucan particles can be prepared by extraction of insoluble components from cell walls, for example by extracting Baker's yeast (Fleischmann's) with 1M NaOH/pH 4.0 H₂O, followed by washing and drying. Methods of preparing yeast cell wall particles are discussed in, and incorporated by reference from U.S. Pat. Nos. 4,810,646, 4,992,540, 5,082,936, 5,028,703, 5,032,401, 5,322,841, 5,401,727, 5,504,079, 5,607,677, 5,968,811, 6,242,594, 6,444,448, 6,476,003, US Patent Publications 2003/0216346, 2004/0014715 and 2010/0040656, and PCT published application WO02/12348.

Protocols for preparing glucan particles are also described in, and incorporated by reference from, the following references: Soto and Ostroff (2008), “Characterization of multilayered nanoparticles encapsulated in yeast cell wall particles for DNA delivery.” Bioconjug Chem 19 (4): 840-8; Soto and Ostroff (2007), “Oral Macrophage Mediated Gene Delivery System,” Nanotech, Volume 2, Chapter 5 (“Drug Delivery”), pages 378-381; and Li et al. (2007), “Yeast glucan particles activate murine resident macrophages to secrete proinflammatory cytokines via MyD88- and Syk kinase-dependent pathways.” Clinical Immunology 124 (2): 170-181.

Glucan containing particles such as yeast cell wall particles can also be obtained commercially. Several non-limiting examples include: Nutricell MOS 55 from Biorigin (Sao Paolo, Brazil), SAF-Mannan (SAF Agri, Minneapolis, Minn.), Nutrex (Sensient Technologies, Milwaukee, Wis.), alkali-extracted particles such as those produced by Nutricepts (Nutricepts Inc., Burnsville, Minn.) and ASA Biotech, acid-extracted WGP particles from Biopolymer Engineering, and organic solvent-extracted particles such as Adjuvax™ from Alpha-beta Technology, Inc. (Worcester, Mass.) and microparticulate glucan from Novogen (Stamford, Conn.).

Glucan particles such as yeast cell wall particles can have varying levels of purity depending on the method of production and/or extraction. In some instances, particles are alkali-extracted, acid-extracted or organic solvent-extracted to remove intracellular components and/or the outer mannoprotein layer of the cell wall. Such protocols can produce particles that have a glucan (w/w) content in the range of 50%-90%. In some instances, a particle of lower purity, meaning lower glucan w/w content may be preferred, while in other embodiments, a particle of higher purity, meaning higher glucan w/w content may be preferred.

Glucan particles, such as yeast cell wall particles, can have a natural lipid content. For example, the particles can contain 1%, 2%, 3%, 4%, 5%, 6%, 7%, 8%, 9%, 10%, 11%, 12%, 13%, 14%, 15%, 16%, 17%, 18%, 19%, 20% or more than 20% w/w lipid. In some instances, the presence of natural lipids may assist in complexation or capture of RNA molecules.

Glucan containing particles typically have a diameter of approximately 2-4 microns, although particles with a diameter of less than 2 microns or greater than 4 microns are also compatible with aspects of the disclosure.

The RNA molecule(s) to be delivered can be complexed or “trapped” within the shell of the glucan particle. The shell or RNA component of the particle can be labeled for visualization, as described in, and incorporated by reference from, Soto and Ostroff (2008) Bioconjug Chem 19:840. Methods of loading GeRPs are discussed further below.

The optimal protocol for uptake of oligonucleotides will depend upon a number of factors, the most crucial being the type of cells that are being used. Other factors that are important in uptake include, but are not limited to, the nature and concentration of the oligonucleotide, the confluence of the cells, the type of culture the cells are in (e.g., a suspension culture or plated) and the type of media in which the cells are grown.

### Carriers and Complexing Agents

The disclosure further relates to compositions comprising RNAi constructs as described herein, and a pharmaceutically acceptable carrier or diluent. In some aspects, the disclosure relates to immunogenic compositions comprising the RNAi constructs described herein, and a pharmaceutically acceptable carrier.

As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

For example, in some embodiments, oligonucleotides may be incorporated into liposomes or liposomes modified with polyethylene glycol or admixed with cationic lipids for parenteral administration. Incorporation of additional substances into the liposome, for example, antibodies reactive against membrane proteins found on specific target cells, can help target the oligonucleotides to specific cell types (e.g., immune cells, such as T cells).

Encapsulating agents entrap oligonucleotides within vesicles. In another embodiment of the disclosure, an oligonucleotide may be associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or toxicologic properties.

For example, the oligonucleotides of the present disclosure may also be administered encapsulated in liposomes, pharmaceutical compositions wherein the active ingredient is contained either dispersed or variously present in corpuscles consisting of aqueous concentric layers adherent to lipidic layers. The oligonucleotides, depending upon solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns.

The use of liposomes as drug delivery vehicles offers several advantages. Liposomes increase intracellular stability, increase uptake efficiency and improve biological activity.

Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a lipid delivery vehicle originally designed as a research tool, such as Lipofectin or LIPOFECTAMINE™ 2000, can deliver intact nucleic acid molecules to cells.

Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

In some aspects, formulations associated with the disclosure might be selected for a class of naturally occurring or chemically synthesized or modified saturated and unsaturated fatty acid residues. Fatty acids might exist in a form of triglycerides, diglycerides or individual fatty acids. In another embodiment, the use of well-validated mixtures of fatty acids and/or fat emulsions currently used in pharmacology for parenteral nutrition may be utilized.

Liposome based formulations are widely used for oligonucleotide delivery. However, most of commercially available lipid or liposome formulations contain at least one positively charged lipid (e.g., a cationic lipid). The presence of this positively charged lipid is believed to be essential for obtaining a high degree of oligonucleotide loading and for enhancing liposome fusogenic properties. Several methods have been performed and published to identify functional positively charged lipid chemistries. However, the commercially available liposome formulations containing cationic lipids are characterized by a high level of toxicity. In vivo limited therapeutic indexes have revealed that liposome formulations containing positive charged lipids are associated with toxicity (e.g., elevation in liver enzymes) at concentrations only slightly higher than concentration required to achieve RNA silencing.

Nucleic acids associated with the disclosure can be hydrophobically modified and can be encompassed within neutral nanotransporters. Further description of neutral nanotransporters is incorporated by reference from PCT Application PCT/US2009/005251, filed on Sep. 22, 2009, and entitled “Neutral Nanotransporters.” Such particles enable quantitative oligonucleotide incorporation into non-charged lipid mixtures. The lack of toxic levels of cationic lipids in such neutral nanotransporter compositions is an important feature.

As demonstrated in PCT/US2009/005251, oligonucleotides can effectively be incorporated into a lipid mixture that is free of cationic lipids and such a composition can effectively deliver a therapeutic oligonucleotide to a cell in a manner that it is functional. For example, a high level of activity was observed when the fatty mixture was composed of a phosphatidylcholine base fatty acid and a sterol such as a cholesterol. For instance, one preferred formulation of neutral fatty mixture is composed of at least 20% of DOPC or DSPC and at least 20% of sterol such as cholesterol. Even as low as 1:5 lipid to oligonucleotide ratio was shown to be sufficient to get complete encapsulation of the oligonucleotide in a non-charged formulation.

The neutral nanotransporters compositions enable efficient loading of oligonucleotide into neutral fat formulation. The composition includes an oligonucleotide that is modified in a manner such that the hydrophobicity of the molecule is increased (for example a hydrophobic molecule is attached (covalently or no-covalently) to a hydrophobic molecule on the oligonucleotide terminus or a non-terminal nucleotide, base, sugar, or backbone), the modified oligonucleotide being mixed with a neutral fat formulation (for example containing at least 25% of cholesterol and 25% of DOPC or analogs thereof). A cargo molecule, such as another lipid can also be included in the composition. This composition, where part of the formulation is built into the oligonucleotide itself, enables efficient encapsulation of oligonucleotide in neutral lipid particles.

In some aspects, stable particles ranging in size from 50 to 140 nm can be formed upon complexing of hydrophobic oligonucleotides with preferred formulations. The formulation by itself typically does not form small particles, but rather, forms agglomerates, which are transformed into stable 50-120 nm particles upon addition of the hydrophobic modified oligonucleotide.

In some embodiments, neutral nanotransporter compositions include a hydrophobic modified polynucleotide, a neutral fatty mixture, and optionally a cargo molecule. A “hydrophobic modified polynucleotide” as used herein is a polynucleotide of the disclosure (e.g., sd-rxRNA) that has at least one modification that renders the polynucleotide more hydrophobic than the polynucleotide was prior to modification. The modification may be achieved by attaching (covalently or non-covalently) a hydrophobic molecule to the polynucleotide. In some instances the hydrophobic molecule is or includes a lipophilic group.

The term “lipophilic group” means a group that has a higher affinity for lipids than its affinity for water. Examples of lipophilic groups include, but are not limited to, cholesterol, a cholesteryl or modified cholesteryl residue, adamantine, dihydrotesterone, long chain alkyl, long chain alkenyl, long chain alkynyl, olely-lithocholic, cholenic, oleoyl-cholenic, palmityl, heptadecyl, myrisityl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g., Cy3 or Cy5), Hoechst 33258 dye, psoralen, or ibuprofen. The cholesterol moiety may be reduced (e.g., as in cholestan) or may be substituted (e.g., by halogen). A combination of different lipophilic groups in one molecule is also possible.

The hydrophobic molecule may be attached at various positions of the polynucleotide. As described above, the hydrophobic molecule may be linked to the terminal residue of the polynucleotide such as the 3′ of 5′-end of the polynucleotide. Alternatively, it may be linked to an internal nucleotide or a nucleotide on a branch of the polynucleotide. The hydrophobic molecule may be attached, for instance to a 2′-position of the nucleotide. The hydrophobic molecule may also be linked to the heterocyclic base, the sugar or the backbone of a nucleotide of the polynucleotide.

The hydrophobic molecule may be connected to the polynucleotide by a linker moiety. Optionally the linker moiety is a non-nucleotidic linker moiety. Non-nucleotidic linkers are e.g. abasic residues (dSpacer), oligoethyleneglycol, such as triethyleneglycol (spacer 9) or hexaethylenegylcol (spacer 18), or alkane-diol, such as butanediol. The spacer units are preferably linked by phosphodiester or phosphorothioate bonds. The linker units may appear just once in the molecule or may be incorporated several times, e.g., via phosphodiester, phosphorothioate, methylphosphonate, or amide linkages.

Typical conjugation protocols involve the synthesis of polynucleotides bearing an aminolinker at one or more positions of the sequence, however, a linker is not required. The amino group is then reacted with the molecule being conjugated using appropriate coupling or activating reagents. The conjugation reaction may be performed either with the polynucleotide still bound to a solid support or following cleavage of the polynucleotide in solution phase. Purification of the modified polynucleotide by HPLC typically results in a pure material.

In some embodiments the hydrophobic molecule is a sterol type conjugate, a PhytoSterol conjugate, cholesterol conjugate, sterol type conjugate with altered side chain length, fatty acid conjugate, any other hydrophobic group conjugate, and/or hydrophobic modifications of the internal nucleoside, which provide sufficient hydrophobicity to be incorporated into micelles.

For purposes of the present disclosure, the term “sterols”, refers or steroid alcohols are a subgroup of steroids with a hydroxyl group at the 3-position of the A-ring. They are amphipathic lipids synthesized from acetyl-coenzyme A via the HMG-COA reductase pathway. The overall molecule is quite flat. The hydroxyl group on the A ring is polar. The rest of the aliphatic chain is non-polar. Usually sterols are considered to have an 8 carbon chain at position 17.

For purposes of the present disclosure, the term “sterol type molecules”, refers to steroid alcohols, which are similar in structure to sterols. The main difference is the structure of the ring and number of carbons in a position 21 attached side chain.

For purposes of the present disclosure, the term “PhytoSterols” (also called plant sterols) are a group of steroid alcohols, phytochemicals naturally occurring in plants. There are more than 200 different known PhytoSterols.

For purposes of the present disclosure, the term “sterol side chain” refers to a chemical composition of a side chain attached at the position 17 of sterol-type molecule. In a standard definition, sterols are limited to a 4 ring structure carrying an 8 carbon chain at position 17. In this disclosure, the sterol type molecules with side chain longer and shorter than conventional are described. The side chain may be branched or contain double back bones.

Thus, sterols useful in the disclosure, for example, include cholesterols, as well as unique sterols in which position 17 has attached side chain of 2-7 or longer than 9 carbons. In some embodiments the length of the polycarbon tail is varied between 5 and 9 carbons. Such conjugates may have significantly better in vivo efficacy, in particular delivery to liver. These types of molecules are expected to work at concentrations 5 to 9 fold lower then oligonucleotides conjugated to conventional cholesterols.

Alternatively the polynucleotide may be bound to a protein, peptide or positively charged chemical that functions as the hydrophobic molecule. The proteins may be selected from the group consisting of protamine, dsRNA binding domain, and arginine rich peptides. Exemplary positively charged chemicals include spermine, spermidine, cadaverine, and putrescine.

In another embodiment hydrophobic molecule conjugates may demonstrate even higher efficacy when it is combined with specific chemical modification patterns of the polynucleotide (as described herein in detail), containing but not limited to hydrophobic modifications, phosphorothioate modifications, and 2′ ribo modifications.

In another embodiment the sterol type molecule may be a naturally occurring PhytoSterols. The polycarbon chain may be longer than 9 and may be linear, branched and/or contain double bonds. Some PhytoSterol-containing polynucleotide conjugates may be significantly more potent and active in delivery of polynucleotides to various tissues. Some PhytoSterols may demonstrate tissue preference and thus be used as a way to delivery RNAi specifically to particular tissues.

The hydrophobic modified polynucleotide is mixed with a neutral fatty mixture to form a micelle. The neutral fatty acid mixture is a mixture of fats that has a net neutral or slightly net negative charge at or around physiological pH that can form a micelle with the hydrophobic modified polynucleotide. For purposes of the present disclosure, the term “micelle” refers to a small nanoparticle formed by a mixture of non-charged fatty acids and phospholipids. The neutral fatty mixture may include cationic lipids as long as they are present in an amount that does not cause toxicity. In some embodiments the neutral fatty mixture is free of cationic lipids. A mixture that is free of cationic lipids is one that has less than 1% and preferably 0% of the total lipid being cationic lipid. The term “cationic lipid” includes lipids and synthetic lipids having a net positive charge at or around physiological pH. The term “anionic lipid” includes lipids and synthetic lipids having a net negative charge at or around physiological pH.

The neutral fats bind to the oligonucleotides of the disclosure by a strong but non-covalent attraction (e.g., an electrostatic, van der Waals, pi-stacking, etc. interaction).

The neutral fat mixture may include formulations selected from a class of naturally occurring or chemically synthesized or modified saturated and unsaturated fatty acid residues. Fatty acids might exist in a form of triglycerides, diglycerides or individual fatty acids. In another embodiment the use of well-validated mixtures of fatty acids and/or fat emulsions currently used in pharmacology for parenteral nutrition may be utilized.

The neutral fatty mixture is preferably a mixture of a choline based fatty acid and a sterol. Choline based fatty acids include for instance, synthetic phosphocholine derivatives such as DDPC, DLPC, DMPC, DPPC, DSPC, DOPC, POPC, and DEPC. DOPC (chemical registry number 4235-95-4) is dioleoylphosphatidylcholine (also known as dielaidoylphosphatidylcholine, dioleoyl-PC, dioleoylphosphocholine, dioleoyl-sn-glycero-3-phosphocholine, dioleylphosphatidylcholine). DSPC (chemical registry number 816-94-4) is distearoylphosphatidylcholine (also known as 1,2-Distearoyl-sn-Glycero-3-phosphocholine).

The sterol in the neutral fatty mixture may be for instance cholesterol. The neutral fatty mixture may be made up completely of a choline based fatty acid and a sterol or it may optionally include a cargo molecule. For instance, the neutral fatty mixture may have at least 20% or 25% fatty acid and 20% or 25% sterol.

For purposes of the present disclosure, the term “fatty acids” relates to conventional description of fatty acid. They may exist as individual entities or in a form of two- and triglycerides. For purposes of the present disclosure, the term “fat emulsions” refers to safe fat formulations given intravenously to subjects who are unable to get enough fat in their diet. It is an emulsion of soybean oil (or other naturally occurring oils) and egg phospholipids. Fat emulsions are being used for formulation of some insoluble anesthetics. In this disclosure, fat emulsions might be part of commercially available preparations like Intralipid, Liposyn, Nutrilipid, modified commercial preparations, where they are enriched with particular fatty acids or fully de novo-formulated combinations of fatty acids and phospholipids.

In one embodiment, the cells to be contacted with an oligonucleotide composition of the disclosure are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

50%-60% of the formulation can optionally be any other lipid or molecule. Such a lipid or molecule is referred to herein as a cargo lipid or cargo molecule. Cargo molecules include but are not limited to intralipid, small molecules, fusogenic peptides or lipids or other small molecules might be added to alter cellular uptake, endosomal release or tissue distribution properties. The ability to tolerate cargo molecules is important for modulation of properties of these particles, if such properties are desirable. For instance the presence of some tissue specific metabolites might drastically alter tissue distribution profiles. For example use of Intralipid type formulation enriched in shorter or longer fatty chains with various degrees of saturation affects tissue distribution profiles of these type of formulations (and their loads).

An example of a cargo lipid useful according to the disclosure is a fusogenic lipid. For instance, the zwiterionic lipid DOPE (chemical registry number 4004 May 1,1,2-Dioleoyl-sn-Glycero-3-phosphoethanolamine) is a preferred cargo lipid.

Intralipid may be comprised of the following composition: 1 000 mL contain: purified soybean oil 90 g, purified egg phospholipids 12 g, glycerol anhydrous 22 g, water for injection q.s. ad 1 000 mL. pH is adjusted with sodium hydroxide to pH approximately 8. Energy content/L: 4.6 MJ (190 kcal). Osmolality (approx.): 300 mOsm/kg water. In another embodiment fat emulsion is Liposyn that contains 5% safflower oil, 5% soybean oil, up to 1.2% egg phosphatides added as an emulsifier and 2.5% glycerin in water for injection. It may also contain sodium hydroxide for pH adjustment. pH 8.0 (6.0-9.0). Liposyn has an osmolarity of 276 m Osmol/liter (actual).

Variation in the identity, amounts and ratios of cargo lipids affects the cellular uptake and tissue distribution characteristics of these compounds. For example, the length of lipid tails and level of saturability will affect differential uptake to liver, lung, fat and cardiomyocytes. Addition of special hydrophobic molecules like vitamins or different forms of sterols can favor distribution to special tissues which are involved in the metabolism of particular compounds. In some embodiments, vitamin A or E is used. Complexes are formed at different oligonucleotide concentrations, with higher concentrations favoring more efficient complex formation.

In another embodiment, the fat emulsion is based on a mixture of lipids. Such lipids may include natural compounds, chemically synthesized compounds, purified fatty acids or any other lipids. In yet another embodiment the composition of fat emulsion is entirely artificial. In a particular embodiment, the fat emulsion is more than 70% linoleic acid. In yet another particular embodiment the fat emulsion is at least 1% of cardiolipin. Linoleic acid (LA) is an unsaturated omega-6 fatty acid. It is a colorless liquid made of a carboxylic acid with an 18-carbon chain and two cis double bonds.

In yet another embodiment of the present disclosure, the alteration of the composition of the fat emulsion is used as a way to alter tissue distribution of hydrophobically modified polynucleotides. This methodology provides for the specific delivery of the polynucleotides to particular tissues.

In another embodiment the fat emulsions of the cargo molecule contain more than 70% of Linoleic acid (C₁₈H₃₂O₂) and/or cardiolipin.

Fat emulsions, like intralipid have been used before as a delivery formulation for some non-water soluble drugs (such as Propofol, re-formulated as Diprivan). Unique features of the present disclosure include (a) the concept of combining modified polynucleotides with the hydrophobic compound(s), so it can be incorporated in the fat micelles and (b) mixing it with the fat emulsions to provide a reversible carrier. After injection into a blood stream, micelles usually bind to serum proteins, including albumin, HDL, LDL and other. This binding is reversible and eventually the fat is absorbed by cells. The polynucleotide, incorporated as a part of the micelle will then be delivered closely to the surface of the cells. After that cellular uptake might be happening though variable mechanisms, including but not limited to sterol type delivery.

Complexing agents bind to the oligonucleotides of the disclosure by a strong but non-covalent attraction (e.g., an electrostatic, van der Waals, pi-stacking, etc. interaction). In one embodiment, oligonucleotides of the disclosure can be complexed with a complexing agent to increase cellular uptake of oligonucleotides. An example of a complexing agent includes cationic lipids. Cationic lipids can be used to deliver oligonucleotides to cells. However, as discussed above, formulations free in cationic lipids are preferred in some embodiments.

The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl⁻, Br⁻, I⁻, F⁻, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N-(N′,N′-dimethylaminoethane) carbamoyl]cholesterol (DC-Chol), 2,3, —dioleyloxy-N-[2 (sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). The cationic lipid N-(1-(2,3-dioleyloxy) propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). Oligonucleotides can also be complexed with, e.g., poly (L-lysine) or avidin and lipids may, or may not, be included in this mixture, e.g., steryl-poly (L-lysine).

Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. Nos. 4,235,871; 4,501,728; 4,837,028; 4,737,323.

In one embodiment lipid compositions can further comprise agents, e.g., viral proteins to enhance lipid-mediated transfections of oligonucleotides (Kamata, et al., 1994. Nucl. Acids. Res. 22:536). In another embodiment, oligonucleotides are contacted with cells as part of a composition comprising an oligonucleotide, a peptide, and a lipid as taught, e.g., in U.S. Pat. No. 5,736,392. Improved lipids have also been described which are serum resistant (Lewis, et al., 1996. Proc. Natl. Acad. Sci. 93:3176). Cationic lipids and other complexing agents act to increase the number of oligonucleotides carried into the cell through endocytosis.

In another embodiment, N-substituted glycine oligonucleotides (peptoids) can be used to improve uptake of oligonucleotides. Peptoids have been used to create cationic lipid-like compounds for transfection (Murphy, et al., 1998. Proc. Natl. Acad. Sci. 95:1517). Peptoids can be synthesized using standard methods (e.g., Zuckermann, R. N., et al. 1992. J. Am. Chem. Soc. 114:10646; Zuckermann, R. N., et al. 1992. Int. J. Peptide Protein Res. 40:497). Combinations of cationic lipids and peptoids, liptoids, can also be used to improve uptake of the subject oligonucleotides (Hunag, et al., 1998. Chemistry and Biology. 5:345). Liptoids can be synthesized by elaborating peptoid oligonucleotides and coupling the amino terminal submonomer to a lipid via its amino group (Hunag, et al., 1998. Chemistry and Biology. 5:345).

It is known in the art that positively charged amino acids can be used for creating highly active cationic lipids (Lewis et al. 1996. Proc. Natl. Acad. Sci. U.S.A. 93:3176). In one embodiment, a composition for delivering oligonucleotides of the disclosure comprises a number of arginine, lysine, histidine or ornithine residues linked to a lipophilic moiety (see e.g., U.S. Pat. No. 5,777,153).

In another embodiment, a composition for delivering oligonucleotides of the disclosure comprises a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably, a preponderance of neutral amino acids with long neutral side chains is used.

In one embodiment, a composition for delivering oligonucleotides of the disclosure comprises a natural or synthetic polypeptide having one or more gamma carboxyglutamic acid residues, or γ-Gla residues. These gamma carboxyglutamic acid residues may enable the polypeptide to bind to each other and to membrane surfaces. In other words, a polypeptide having a series of γ-Gla may be used as a general delivery modality that helps an RNAi construct to stick to whatever membrane to which it comes in contact. This may slow RNAi constructs from being cleared from the blood stream and enhance their chance of homing to the target.

The gamma carboxyglutamic acid residues may exist in natural proteins (for example, prothrombin has 10 γ-Gla residues). Alternatively, they can be introduced into the purified, recombinantly produced, or chemically synthesized polypeptides by carboxylation using, for example, a vitamin K-dependent carboxylase. The gamma carboxyglutamic acid residues may be consecutive or non-consecutive, and the total number and location of such gamma carboxyglutamic acid residues in the polypeptide can be regulated/fine-tuned to achieve different levels of “stickiness” of the polypeptide.

In one embodiment, the cells to be contacted with an oligonucleotide composition of the disclosure are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

For example, in one embodiment, an oligonucleotide composition can be contacted with cells in the presence of a lipid such as cytofectin CS or GSV (available from Glen Research; Sterling, Va.), GS3815, GS2888 for prolonged incubation periods as described herein.

In one embodiment, the incubation of the cells with the mixture comprising a lipid and an oligonucleotide composition does not reduce the viability of the cells. Preferably, after the transfection period the cells are substantially viable. In one embodiment, after transfection, the cells are between at least about 70% and at least about 100% viable. In another embodiment, the cells are between at least about 80% and at least about 95% viable. In yet another embodiment, the cells are between at least about 85% and at least about 90% viable.

In one embodiment, oligonucleotides are modified by attaching a peptide sequence that transports the oligonucleotide into a cell, referred to herein as a “transporting peptide.” In one embodiment, the composition includes an oligonucleotide which is complementary to a target nucleic acid molecule encoding the protein, and a covalently attached transporting peptide.

The language “transporting peptide” includes an amino acid sequence that facilitates the transport of an oligonucleotide into a cell. Exemplary peptides which facilitate the transport of the moieties to which they are linked into cells are known in the art, and include, e.g., HIV TAT transcription factor, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88:223).

Oligonucleotides can be attached to the transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J. Biol. Chem. 272:16010). For example, in one embodiment, oligonucleotides bearing an activated thiol group are linked via that thiol group to a cysteine present in a transport peptide (e.g., to the cysteine present in the β turn between the second and the third helix of the antennapedia homeodomain as taught, e.g., in Derossi et al. 1998. Trends Cell Biol. 8:84; Prochiantz. 1996. Current Opinion in Neurobiol. 6:629; Allinquant et al. 1995. J Cell Biol. 128:919). In another embodiment, a Boc-Cys-(Npys) OH group can be coupled to the transport peptide as the last (N-terminal) amino acid and an oligonucleotide bearing an SH group can be coupled to the peptide (Troy et al. 1996. J. Neurosci. 16:253).

In one embodiment, a linking group can be attached to a nucleomonomer and the transporting peptide can be covalently attached to the linker. In one embodiment, a linker can function as both an attachment site for a transporting peptide and can provide stability against nucleases. Examples of suitable linkers include substituted or unsubstituted C₁-C₂₀ alkyl chains, C₂-C₂₀ alkenyl chains, C₂-C₂₀ alkynyl chains, peptides, and heteroatoms (e.g., S, O, NH, etc.). Other exemplary linkers include bifunctional crosslinking agents such as sulfosuccinimidyl-4-(maleimidophenyl)-butyrate (SMPB) (see, e.g., Smith et al. Biochem J 1991.276:417-2).

In one embodiment, oligonucleotides of the disclosure are synthesized as molecular conjugates which utilize receptor-mediated endocytotic mechanisms for delivering genes into cells (see, e.g., Bunnell et al. 1992. Somatic Cell and Molecular Genetics. 18:559, and the references cited therein).

Other carriers for in vitro and/or in vivo delivery of RNAi reagents are known in the art, and can be used to deliver the subject RNAi constructs (e.g., to an NK cell). See, for example, U.S. patent application publications 20080152661, 20080112916, 20080107694, 20080038296, 20070231392, 20060240093, 20060178327, 20060008910, 20050265957, 20050064595, 20050042227, 20050037496, 20050026286, 20040162235, 20040072785, 20040063654, 20030157030, WO 2008/036825, WO04/065601, and AU2004206255B2, just to name a few (all incorporated by reference).

### Immunogenic Compositions and Methods of Producing the Same

In some embodiments, chemically modified double stranded nucleic acid molecules (e.g., INTASYL™ molecules) described herein are useful for producing specific cell subtypes or T cell subtypes for immunogenic compositions. As used herein, an “immunogenic composition” is a composition comprising a host cell comprising a chemically modified nucleic acid molecule as described herein, and optionally one or more pharmaceutically acceptable excipients or carriers. Without wishing to be bound by any particular theory, immunogenic compositions as described by the disclosure are characterized by a population of immune cells (e.g., T cells) that have been engineered to have an enriched population of a particular cell subtype (e.g., T cell subtype, such as stem-like T cells) and/or reduced (e.g., inhibited) expression or activity of one or more immune checkpoint proteins (e.g., PD-1), and are thus useful, in some embodiments, for modulating (e.g., stimulating or inhibiting) the immune response of a subject.

As used herein, a “host cell” is a cell to which one or more chemically modified double stranded nucleic acid molecules have been introduced. Typically, a host cell is a mammalian cell, for example a human cell, mouse cell, rat cell, pig cell, etc. However, in some embodiments, a host cell is a non-mammalian cell, for example a prokaryotic cell (e.g., bacterial cell), yeast cell, insect cell, etc. Generally, a host cell is derived from a donor, such as a healthy donor (e.g., the cell to which the chemically modified double stranded nucleic acid is introduced is taken from a donor, such as a healthy donor). For example, a cell or cells may be isolated from a biological sample obtained from a donor, such as a healthy donor, such as bone marrow or blood. As used herein “healthy donor” refers to a subject that does not have, or is not suspected of having, a proliferative disorder or an infectious disease (e.g., a bacterial, viral, or parasitic infection). However, in some embodiments, a host cell is derived from a subject having (or suspected of having) a proliferative disease or an infectious disease, for example in the context of autologous cell therapy.

In some embodiments, a cell (e.g., a host cell) is an immune cell, for example a T cell. In some embodiments, a cell (e.g., a host cell) is a T cell, such as a killer T cell, helper T cell, or a regulatory T cell. In some embodiments, the T cell is a killer T cell (e.g., a CD8+ T-cell). As used herein, “CD8+ T cell” refers to a T cell expressing a heterodimeric co-receptor, CD8. CD8 typically comprises one CD8α and one CD8β chain. CD8+ T cells recognize peptides presented by MHC Class I molecules, found on all nucleated cells. The CD8 heterodimer binds to a conserved portion (the α3 region) of MHC Class I molecule during T cell/antigen presenting cell interactions, activating the CD8+ T cell and resulting in the elimination of the target cell. In some embodiments, a T cell is a double-negative T cell (e.g., CD8−, CD4− T cell). In some embodiments, a T cell is an activated T cell (e.g., a T cell that has been presented with a peptide antigen by MHC class I molecules on an antigen presenting cell). In some embodiments, a T cell is a memory T cell. In some embodiments, a T cell is a naïve T cell.

In some embodiments, a T cell comprises one or more transgenes expressing a high affinity T-cell receptor (TCR) and/or a chimeric antibody receptor (CAR). In some embodiments, the TCR is a TCRαβ heterodimer.

In some embodiments, the T cell is a tumor infiltrating lymphocyte (TIL).

In some aspects, the disclosure relates to the discovery that introducing one or more chemically modified double stranded nucleic acid molecules of the disclosure to a cell (e.g., an immune cell obtained from a donor) to produce a host cell results in a significant reduction of immune checkpoint protein (e.g., PD-1) expression or activity in the host cell. In some embodiments, a host cell is characterized by between about 5% and about 50%, about 5% and about 40%, about 5% and about 30%, about 5% and about 20%, about 5% and about 10%, about 10% and about 50%, about 10% and about 40%, about 10% and about 30%, about 10% and about 20%, about 15% and about 50%, about 15% and about 40%, about 15% and about 30%, about 15% and about 20%, about 20% and about 50%, about 20% and about 40%, about 20% and about 30%, about 25% and about 50%, about 25% and about 40%, about 25% and about 30%, about 30% and about 50%, about 30% and about 40%, about 35% and about 50%, about 35% and about 40%, about 40% and about 50%, and about 45% and about 50% reduced expression of an immune checkpoint protein relative to a cell (e.g., an immune cell of the same cell type) that does not comprise the chemically modified double stranded nucleic acid molecules. For example, in some embodiments, a host cell is characterized by about 5%, 10%, 15%, 20%, 25%, 30%, 35%, 40%, 50% or more reduced expression of an immune checkpoint protein relative to a cell (e.g., an immune cell of the same cell type) that does not comprise the chemically modified double stranded nucleic acid molecules. In some embodiments, a host cell is characterized by greater than 50% (e.g., 51%, 52%, 53%, 54%, 55%, 60%, 70%, 75%, 80%, 85%, 90%, 95%, 99%, 100%, or about any percentage between 51% and 100%) reduced expression of an immune checkpoint protein relative to a cell (e.g., a naïve cell, such as an immune cell of the same cell type) that does not comprise the chemically modified double stranded nucleic acid molecules (e.g., an immune cell of a subject having or suspected of having a proliferative disease).

In some embodiments, an immunogenic composition as described by the disclosure comprises a plurality of host cells. In some embodiments, the plurality of host cells is about 10,000 host cells per kilogram, about 50,000 host cells per kilogram, about 100,000 host cells per kilogram, about 250,000 host cells per kilogram, about 500,000 host cells per kilogram, about 1×10⁶ host cells per kilogram, about 5×10⁶ host cells per kilogram, about 1×10⁷ host cells per kilogram, about 1×10⁸ host cells per kilogram, about 1×10⁹ host cells per kilogram, or more than 1×10⁹ host cells per kilogram. In some embodiments, the plurality of host cells is between about 1×10⁵ and 1×10¹⁴ host cells per kilogram.

In some aspects, the disclosure provides methods for producing an immunogenic composition as described by the disclosure. In some embodiments, the methods comprise, introducing into a cell one or more chemically modified double stranded nucleic acid molecules (e.g., INTASYL™ molecules), wherein the one or more chemically modified double stranded nucleic acid molecules target PD-1, thereby producing a host cell with a specific cell subtype or T cell subtype (e.g., stem-like T cell). As used herein, “stem-like T cell” (e.g., stem-like CD8+ T cell) refers to a T cell that comprises at least one stem-like surface marker (e.g., is TCF-1-positive). In some embodiments, stem-like T cells also express CD95, CD45RA, CCR7, CD27, CD127, CD28, T-box expressed in T cells (T-bet), KI-67 and/or TOX. Stem-like T cells are capable of multiplying, differentiating, and/or self-renewing. For example, during proliferation, stem-like CD8+ T cells yield more terminally differentiated, effector-molecule-expressing daughter cells. Stem-like CD8+ T cells also sustain antigen-specific CD8+ T cell responses during chronic antigen exposure. In some embodiments, the introduction of one or more chemically modified double stranded nucleic acid molecules (e.g., INTASYL™ molecules), wherein the one or more chemically modified double stranded nucleic acid molecules target PD-1 yields a population of T cells, including at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, or at least 30% stem-like T cells (e.g., stem-like CD8+ T cells). In some embodiments, an immunogenic composition comprising T cells and one or more chemically modified double stranded nucleic acid molecules (e.g., INTASYL™ molecules) targeting PD-1 yields a population of T cells having a greater proportion of stem-like CD8+ T cells than an untreated population of T cells (e.g., a population of T cells that has not been exposed to one or more chemically modified double stranded nucleic acid molecules (e.g., INTASYL™ molecules) targeting PD-1). In some embodiments, the stem-like T cells (e.g., stem-like CD8+ T cells) express TCF-1. In some embodiments, the stem-like T cells (e.g., stem-like CD8+ T cells) express KI-67. In some embodiments, the stem-like T cells (e.g., stem-like CD8+ T cells) express TOX. In some embodiments, the stem-like T cells (e.g., stem-like CD8+ T cells) express CD95. In some embodiments, the stem-like T cells (e.g., stem-like CD8+ T cells) express CD45RA, In some embodiments, the stem-like T cells (e.g., stem-like CD8+ T cells) express CCR7. In some embodiments, the stem-like T cells (e.g., stem-like CD8+ T cells) express CD27. In some embodiments, the stem-like T cells (e.g., stem-like CD8+ T cells) express CD127. In some embodiments, the stem-like T cells (e.g., stem-like CD8+ T cells) express CD28, In some embodiments, the stem-like T cells (e.g., stem-like CD8+ T cells) express T-bet. In some embodiments, the stem-like T cells (e.g., stem-like CD8+ T cells) express TCF-1 and at least one of the following: CD95, CD45RA, CCR7, CD27, CD127, CD28, T-bet, KI-67, and TOX. In some embodiments, the stem-like T cells (e.g., stem-like CD8+ T cells) express TCF-1 and at least two of the following: CD95, CD45RA, CCR7, CD27, CD127, CD28, T-bet, KI-67, and TOX. In some embodiments, the stem-like T cells (e.g., stem-like CD8+ T cells) express TCF-1 CD95, CD45RA, CCR7, CD27, CD127, CD28, T-bet, KI-67, and TOX.

Methods of producing immunogenic compositions (e.g., producing a host cell or populations of host cells) may be carried out in vitro, ex vivo, or in vivo, in, for example, mammalian cells in culture, such as a human cell in culture. In some embodiments, target cells (e.g., cells obtained from a donor) may be contacted in the presence of a delivery reagent, such as a lipid (e.g., a cationic lipid) or a liposome to facilitate entry of the chemically modified double stranded nucleic acid molecules into the cell, as described in further detail elsewhere in the disclosure.

### Therapeutic Methods

In some aspects, the disclosure provides methods of treating a proliferative disease or an infectious disease by administering to a subject (e.g., a subject having or suspected of having a proliferative disease or an infectious disease) an immunogenic composition as described by the disclosure (e.g., an immunogenic composition comprising one or more host cells of a particular T cell subtype). In some embodiments, immunogenic compositions as described herein are characterized as a population of immune cells (e.g., T cells) having reduced (e.g., inhibited) expression or activity of one or more genes associated with controlling the differentiation process of T cells (e.g., PD-1). Without wishing to be bound by any particular theory, immunogenic compositions as described herein are characterized, in some embodiments, by reduced expression of immune checkpoint proteins and are thus useful for stimulating the immune system of a subject having certain proliferative diseases or infectious diseases characterized by increased expression of immune checkpoint proteins.

As used herein, a “proliferative disease” refers to diseases and disorders characterized by excessive proliferation of cells and turnover of cellular matrix, including cancer, atherlorosclerosis, rheumatoid arthritis, psoriasis, idiopathic pulmonary fibrosis, scleroderma, cirrhosis of the liver, etc. Examples of cancers include but are not limited to small cell lung cancer, colon cancer, breast cancer, lung cancer, prostate cancer, ovarian cancer, kidney cancer, pancreatic cancer, melanoma, bone cancer (e.g., osteosarcoma, etc.), hematological malignancy such as chronic myeloid leukemia (CML), etc.

As used herein, the term “infectious disease” refers to diseases and disorders that result from infection of a subject with a pathogen. Examples of human pathogens include but are not limited to certain bacteria (e.g., certain strains of E. coli, Salmonella, etc.), viruses (HIV, HCV, influenza, etc.), parasites (protozoans, helminths, amoeba, etc.), yeasts (e.g., certain (Candida species, etc.), and fungi (e.g., certain Aspergillus species).

Examples of subjects include mammals, e.g., humans and other primates; cows, pigs, horses, and farming (agricultural) animals; dogs, cats, and other domesticated pets; mice, rats, and transgenic non-human animals.

In some embodiments, immunogenic compositions as described by the disclosure are administered to a subject by ACT therapeutic methods. Examples of ACT modalities include but are not limited to autologous cell therapy (e.g., a subject's own cells are removed, genetically-modified, and returned to the subject) and heterologous cell therapy (e.g., cells are removed from a donor, genetically-modified, and placed into a recipient). In some embodiments, cells utilized in ACT therapeutic methods may be genetically-modified to express chimeric antigen receptors (CARs), which are chimeric proteins resembling antibodies with T cell receptor capacity that are engineered to display specificity against a target antigen based on a selected antibody moiety or engineered to express T cell receptors. Accordingly, in some embodiments, CAR T cells (e.g. CARTs) or TCR T cells may be transfected with a chemically modified double stranded nucleic acid using methods described herein for the purpose of ACT therapy.

With respect to in vivo applications, the formulations of the present disclosure can be administered to a patient in a variety of forms adapted to the chosen route of administration, e.g., parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, or dextran, optionally, the suspension may also contain stabilizers. The oligonucleotides of the disclosure can be formulated in liquid solutions, preferably in physiologically compatible buffers such as Hank's solution or Ringer's solution. In addition, the oligonucleotides may be formulated in solid form and redissolved or suspended immediately prior to use. Lyophilized forms are also included in the disclosure.

Drug delivery vehicles can be chosen e.g., for in vitro, for systemic administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

Administration of an active amount of an oligonucleotide of the present disclosure is defined as an amount effective, at dosages and for periods of time necessary to achieve the desired result. For example, an active amount of an oligonucleotide may vary according to factors such as the type of cell, the oligonucleotide used, and for in vivo uses the disease state, age, sex, and weight of the individual, and the ability of the oligonucleotide to elicit a desired response in the individual. Establishment of therapeutic levels of oligonucleotides within the cell is dependent upon the rates of uptake and efflux or degradation. Decreasing the degree of degradation prolongs the intracellular half-life of the oligonucleotide. Thus, chemically modified oligonucleotides, e.g., with modification of the phosphate backbone, may require different dosing.

The exact dosage of an immunogenic composition and number of doses administered will depend upon the data generated experimentally and in clinical trials. Several factors such as the desired effect, the delivery vehicle, disease indication, and the route of administration, will affect the dosage. Dosages can be readily determined by one of ordinary skill in the art and formulated into the subject pharmaceutical compositions. Preferably, the duration of treatment will extend at least through the course of the disease symptoms.

Dosage regimens may be adjusted to provide the optimum therapeutic response. For example, the immunogenic composition may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject chemically modified double stranded nucleic acid molecules or immunogenic compositions, whether they are to be administered to cells or to subjects.

Administration of immunogenic compositions, such as through intradermal injection or subcutaneous delivery, can be optimized through testing of dosing regimens. In some embodiments, a single administration is sufficient. To further prolong the effect of the administered immunogenic compositions, the compositions can be administered in a slow-release formulation or device, as would be familiar to one of ordinary skill in the art.

In other embodiments, the chemically modified double stranded nucleic acid molecules or immunogenic compositions is administered multiple times. In some instances it is administered daily, bi-weekly, weekly, every two weeks, every three weeks, monthly, every two months, every three months, every four months, every five months, every six months or less frequently than every six months. In some instances, it is administered multiple times per day, week, month and/or year. For example, it can be administered approximately every hour, 2 hours, 3 hours, 4 hours, 5 hours, 6 hours, 7 hours, 8 hours, 9 hours 10 hours, 12 hours or more than twelve hours. It can be administered 1, 2, 3, 4, 5, 6, 7, 8, 9, 10 or more than 10 times per day.

Aspects of the disclosure relate to administering immunogenic compositions to a subject. In some instances the subject is a patient and administering the immunogenic composition involves administering the composition in a doctor's office.

In some embodiments, more than one immunogenic composition is administered simultaneously. For example, in some embodiments, 1, 2, 3, 4, 5, 6, 7, 8, 9, 10 or more than 10 different compositions may be administered. In certain embodiments, a composition comprises 2 or 3 different immunogenic compositions.

In some embodiments, one or more anticancer agents is administered to a subject in combination with one or more immunogenic compositions as described by the disclosure. An “anticancer agent” can be a small molecule, nucleic acid, protein, peptide, polypeptide (e.g., antibody, antibody fragment, etc.), or any combination of the foregoing. In some embodiments, an anticancer agent is administered to the subject prior to administration of the immunogenic composition. In some embodiments, an anticancer agent is administered to a subject after administration of the immunogenic composition. In some embodiments, an anticancer agent is administered concurrently (e.g., at the same time as) with an immunogenic composition.

Examples of anticancer agents include but are not limited to Abitrexate (Methotrexate), Abraxane (Paclitaxel Albumin-stabilized Nanoparticle Formulation), Ado-Trastuzumab Emtansine, Adriamycin PFS (Doxorubicin Hydrochloride), Adriamycin RDF (Doxorubicin Hydrochloride), Adrucil (Fluorouracil), Afinitor (Everolimus), Anastrozole, Aredia (Pamidronate Disodium), Arimidex (Anastrozole), Aromasin (Exemestane), CapecitabineClafen (Cyclophosphamide), Cyclophosphamide, Cytoxan (Cyclophosphamide), Docetaxel, Doxorubicin Hydrochloride, Efudex (Fluorouracil), Ellence (Epirubicin Hydrochloride), Epirubicin Hydrochloride, Everolimus, Exemestane, Fareston (Toremifene), Faslodex (Fulvestrant), Femara (Letrozole), Fluoroplex (Fluorouracil), Fluorouracil, Folex (Methotrexate), Folex PFS (Methotrexate), Fulvestrant, Gemcitabine Hydrochloride, Gemzar (Gemcitabine Hydrochloride), Goserelin Acetate, Herceptin (Trastuzumab), Ixabepilone, Ixempra (Ixabepilone), Kadcyla (Ado-Trastuzumab Emtansine), Lapatinib Ditosylate, Letrozole, Megace (Megestrol Acetate), Megestrol Acetate, Methotrexate, Methotrexate LPF (Methotrexate), Mexate (Methotrexate), Mexate-AQ (Methotrexate), Neosar (Cyclophosphamide), Nolvadex (Tamoxifen Citrate), Novaldex (Tamoxifen Citrate), Paclitaxel, Paclitaxel Albumin-stabilized Nanoparticle Formulation, Pamidronate Disodium, Perjeta (Pertuzumab), Pertuzumab, Tamoxifen Citrate, Taxol (Paclitaxel), Taxotere (Docetaxel), Trastuzumab, Toremifene, Tykerb (Lapatinib Ditosylate), Xeloda (Capecitabine), and Zoladex (Goserelin Acetate).

### Self-Delivering RNAi Immunotherapeutic Agents

As described in U.S. Patent Publication No. US 2016/0304873, the entire contents of which are incorporated herein by reference, immunotherapeutic agents were produced by treating cells with particular sd-rxRNA agents designed to target and knock down specific genes involved in immune suppression mechanisms.

For the purposes of the disclosure, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Moreover, for the purposes of the present disclosure, the term “a” or “an” entity refers to one or more of that entity; for example, “a protein” or “a nucleic acid molecule” refers to one or more of those compounds or at least one compound. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures (i.e., combinations) of two or more of the compounds.

According to the present disclosure, an isolated, or biologically pure, protein or nucleic acid molecule is a compound that has been removed from its natural milieu. As such, “isolated” and “biologically pure” do not necessarily reflect the extent to which the compound has been purified. An isolated compound of the present disclosure can be obtained from its natural source, can be produced using molecular biology techniques or can be produced by chemical synthesis.

Compositions and methods described herein are further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1: Generation of Stem-Like CD8+ T Cell Population

The effect of PD-1 silencing on T cell differentiation in the context of antigenic challenge was studied. TCR53-transduced T cells, suitable for adoptive cell transfer (ACT), were incubated with PD-1 targeting INTASYL™ compound (PD1-11) at a concentration of 2×10⁵ cells/well for 24 hours in 96-well plates. As controls, cells were either treated with a non-targeting compound (NTC) or left untreated (untreated control, UTC). Following 24 hours of INTASYL™ compound loading, T cells were harvested for flow cytometry analysis and for subsequent co-culture with their cognate tumor cell line, RCC-53, for 96 hours at an E:T (effector: target) ratio of 1:2.5. The T cells were harvested and analyzed by flow cytometry using a 10-marker panel including T-Bet, Eomes, TCF-1, Ki67, TOX, PD-1 CD127, KLRG1, perforin, CD8 and CD45 (the latter to exclude remaining tumor cells).

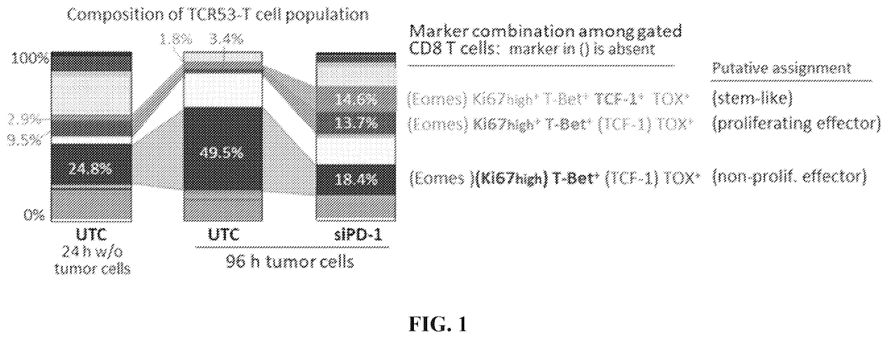

Silencing of PD-1 was found to change the composition of the T cell population that developed during antigenic co-culture (FIG. 1). The untreated control (UTC) TCR53-T cells enriched for a T cell subset that lacked TCF-1, but co-expressed T-Bet with TOX (49.5%, FIG. 1). The enrichment was absent when TCR53-T cells were silenced for PD-1 (siPD-1). Instead, the siPD-1/TCR53 T cells developed a T cell subset that co-expressed T-Bet with TCF-1 as well as Ki67 (14.6%, FIG. 1), which is thought to have the attributes of stem-like T cells. Additionally, it was observed that a putative proliferating effector cell population (T-Bet+Ki67high+without TCF-1) was enriched in PD-1-silenced TCR53-T cells after co-culture (FIG. 1).

TABLE 1
INTASYL ™ sequences targeting PD1

| Duplex | Start | | Seq |
|---|---|---|---|
| ID | Site | Sequence and Chemistry | ID |
| PD1-1 | 2057 | fU.mG.fU.mG.fG.mU.fU.mC.fU.mA.fU.mU.fA*mU*fA.TEG-Chl | 1 |
| | | VP.mU*A*mU.mA.mA.fU.mA.mG.mA.fA.mC.mC.mA.fC*mA*fG*mG*mG*mA*mA. | 2 |
| PD1-2 | 2057 | fU.mG.fU.mG.fG.mU.fU.mC.fU.mA.fU.mU.fA*mU*fA.TEG-Chl | 1 |
| | | VP.mU*mA*mU.fA.mA.fU.mA.mG.mA.fA.mC.mC.mA.mC*mA*fG*mG*mG*mA*mA | 3 |
| PD1-3 | 2057 | fU.mG.fU.mG.fG.mU.fU.mC.fU.mA.fU.mU.fA*mU*fA.TEG-Chl | 1 |
| | | VP.mU*fA*mU.mA.mA.fU.mA.mG.mA.A.mC.mC.mA.fC*mA*fG*mG*mG*mA*mA. | 4 |
| PD1-4 | 2057 | fU.mG.fU.mG.fG.mU.fU.mC.fU.mA.fU.mU.fA*mU*fA.TEG-Chl | 1 |
| | | VP.mU*fA*mU.fA.mA.fU.mA.G.mA.fA.mC.fC.A.mC*mA*fG*mG*mG*mA*mA. | 5 |
| PD1-5 | 2057 | fU.mG.fU.mG.fG.mU.fU.mC.fU.mA.fU.mU.fA*mU*fA.TEG-Chl | 1 |
| | | VP.mU*fA*mU.fA.mA.fU.mA.G.mA.fA.mC.fC.A.fC*mA*fG*mG*mG*mA*mA. | 6 |
| PD1-6 | 2057 | fU.mG.fU.mG.fG.mU.fU.mC.fU.mA.fU.mU.fA*mU*fA.TEG-Chl | 1 |
| | | VP.mU*A*mU.mA.mA.fU.mA.mG.mA.A.fC.mC.mA.fC*mA*fG*mG*mG*mA*mA. | 7 |
| PD1-7 | 2057 | fU.mG.fU.mG.fG.mU.fU.mC.fU.mA.fU.mU.fA*mU*fA.TEG-Chl | 1 |
| | | VP.mU*fA*mU.fA.mA.fU.mA.fG.mA.fA.mC.mC.mA.fC*mA*mG*mG*mG*mA*mA. | 8 |
| PD1-8 | 2057 | mU*mG*fU.fG.fG.mU.fU.mC.mU.mA.fU.mU.mA*mU*mA.TEG-Chl | 9 |
| | | P.mU.fA.fU.mA.mA.fU.mA.mG.mA.fA.fC.fC.mA.fC*mA*mG*mG*mG*mA*mA. | 10 |
| PD1-9 | 2057 | mU*mG*fU.fG.fG.mU.mU.mC.mU.mA.fU.fU.mA*mU*mA.TEG-Chl | 11 |
| | | P.mU.fA.fU.mA.mA.fU.mA.mG.mA.fA.fC.fC.mA.fC*mA*mG*mG*mG*mA*mA. | 10 |
| PD1-10 | 2057 | mU*mG*fU.mG.fG.mU.fU.mC.mU.mA.fU.mU.mA*mU*mA.TEG-Chl | 12 |
| | | P.mU.fA.fU.mA.mA.fU.mA.mG.mA.fA.fC.fC.mA.fC*mA*mG*mG*mG*mA*mA. | 10 |
| PD1-11 | 2057 | fU.mG.fU.mG.fG.mU.fU.mC.fU.mA.fU.mU.fA*mU*fA.TEG-Chl | 1 |
| | | P.mU.fA.fU.m.A.mA.fU.mA.mG.mA.fA.fC.fC.mA.fC*mA*mG*mG*mG*mA*mA. | 10 |

Key
A = adenosine
G = guanosine
U = uridine
C = cytodine
m = 2′-O-methyl nucleotide
f = 2′fluoro nucleotide
Y = 5 methyl uridine
X = 5 methyl cytodine
* = phosphorothioate linkage
. = phosphodiester linkage
TEG-Chl = cholesterol-TEG-Glyceryl
P = 5′ inorganic Phosphate
VP-5′Vinyl Phosphonate
S-5′ Thiophosphate

### EQUIVALENTS

Those skilled in the art will recognize, or be able to ascertain using no more than routine experimentation, many equivalents to the specific embodiments of the disclosure described herein. Such equivalents are intended to be encompassed by the following claims.

What is claimed is:

1. An immunogenic composition comprising a T cell comprising a chemically modified double stranded nucleic acid molecule that is directed against a programmed cell death 1 (PD-1) gene, wherein the chemically modified double stranded nucleic acid molecule comprises at least 12 contiguous nucleotides of a sequence selected from SEQ ID NOs: 1-12.

2. The immunogenic composition of claim 1, wherein the chemically modified double stranded nucleic acid molecule comprises a sequence selected from SEQ ID NOs: 1-12.

3. The immunogenic composition of claim 1 or 2, wherein the chemically modified double stranded nucleic acid molecule is an INTASYL™.

4. The immunogenic composition of claim 3, wherein the INTASYL™ is hydrophobically modified.

5. The immunogenic composition of claim 4, wherein the INTASYL™ is linked to one or more hydrophobic conjugates.

6. The immunogenic composition of any one of claims 1 to 5, wherein the chemically modified double stranded nucleic acid molecule comprises at least one 2′-O-methyl modification and/or at least one 2′-O-Fluoro modification, and at least one phosphorothioate modification.

7. The immunogenic composition of any one of claims 1 to 6, wherein the T cell comprises one or more transgenes expressing a chimeric antibody receptor (CAR) or T cell receptor (TCR).

8. The immunogenic composition of any one of claims 1 to 6, wherein the T cell comprises a tumor infiltrating lymphocyte (TIL).

9. The immunogenic composition of any one of claims 1 to 7, wherein the T cell is derived from a healthy donor or a patient.

10. The immunogenic composition of any one of claims 1 to 8, wherein the chemically modified double stranded nucleic acid molecule induces at least 40% inhibition of PD-1 in the T cell or about 40% inhibition of PD-1 in the T cell.

11. The immunogenic composition of any one of claims 1 to 10, wherein the chemically modified double stranded nucleic acid molecule comprises a sense strand having the sequence set forth in SEQ ID NO: 1 and/or an antisense strand having the sequence set forth in SEQ ID NO: 2 (PD1-1).

12. The immunogenic composition of any one of claims 1 to 10, wherein the chemically modified double stranded nucleic acid molecule comprises a sense strand having the sequence set forth in SEQ ID NO: 1 and/or antisense strand having the sequence set forth in SEQ ID NO: 3 (PD1-2).

13. The immunogenic composition of any one of claims 1 to 10, wherein the chemically modified double stranded nucleic acid molecule comprises a sense strand having the sequence set forth in SEQ ID NO: 1 and/or an antisense strand having the sequence set forth in SEQ ID NO: 4 (PD1-3).

14. The immunogenic composition of any one of claims 1 to 10, wherein the chemically modified double stranded nucleic acid molecule comprises a sense strand having the sequence set forth in SEQ ID NO: 1 and/or an antisense strand having the sequence set forth in SEQ ID NO: 5 (PD1-4).

15. The immunogenic composition of any one of claims 1 to 10, wherein the chemically modified double stranded nucleic acid molecule comprises a sense strand having the sequence set forth in SEQ ID NO: 1 and/or an antisense strand having the sequence set forth in SEQ ID NO: 6 (PD1-5).

16. The immunogenic composition of any one of claims 1 to 10, wherein the chemically modified double stranded nucleic acid molecule comprises a sense strand having the sequence set forth in SEQ ID NO: 1 and/or an antisense strand having the sequence set forth in SEQ ID NO: 7 (PD1-6).

17. The immunogenic composition of any one of claims 1 to 10, wherein the chemically modified double stranded nucleic acid molecule comprises a sense strand having the sequence set forth in SEQ ID NO: 1 and/or an antisense strand having the sequence set forth in SEQ ID NO: 8 (PD1-7).

18. The immunogenic composition of any one of claims 1 to 10, wherein the chemically modified double stranded nucleic acid molecule comprises a sense strand having the sequence set forth in SEQ ID NO: 9 and/or an antisense strand having the sequence set forth in SEQ ID NO: 10 (PD1-8).

19. The immunogenic composition of any one of claims 1 to 10, wherein the chemically modified double stranded nucleic acid molecule comprises a sense strand having the sequence set forth in SEQ ID NO: 11 and/or an antisense strand having the sequence set forth in SEQ ID NO: 10 (PD1-9).

20. The immunogenic composition of any one of claims 1 to 10, wherein the chemically modified double stranded nucleic acid molecule comprises a sense strand having the sequence set forth in SEQ ID NO: 12 and/or an antisense strand having the sequence set forth in SEQ ID NO: 10 (PD1-10).

21. The immunogenic composition of any one of claims 1 to 10, wherein the chemically modified double stranded nucleic acid molecule comprises a sense strand having the sequence set forth in SEQ ID NO: 1 and/or an antisense strand having the sequence set forth in SEQ ID NO: 10 (PD1-11).

22. The immunogenic composition of any one of claims 1 to 21, wherein the T cell is a CD8+ T cell.

23. The immunogenic composition of claim 22, wherein the T cell has a stem-like T cell phenotype.

24. The immunogenic composition of claim 23, wherein the T cell expresses T cell factor 1 (TCF-1).

25. The immunogenic composition of claim 24, wherein the T cell further expresses CD95, CD45RA, CCR7, CD27, CD127, CD28, T-box expressed in T cells (T-bet), KI-67, thymocyte selection associated high mobility group box (TOX), or any combination thereof.

26. The immunogenic composition of any one of claims 1 to 25, further comprising a population of T cells and chemically modified double stranded nucleic acid molecules that are directed against a programmed cell death 1 (PD-1) gene, wherein the chemically modified double stranded molecules comprise at least 12 contiguous nucleotides of a sequence selected from SEQ ID NOs: 1-12.

27. The immunogenic composition of claim 26, wherein the population of T cells has a greater proportion of stem-like CD8+ T cells than an untreated population of T cells.

28. The immunogenic composition of claims 26 to 27, wherein the immunogenic composition comprises at least 5%, at least 10%, or at least 15% stem-like CD8+ T cells.

29. A method for producing a composition comprising stem-like T cells, the method comprising introducing one or more chemically modified double stranded nucleic molecules targeting PD-1 into T cells, wherein the chemically modified double stranded molecule comprises at least 12 contiguous nucleotides of a sequence selected from SEQ ID NOs: 1-12, thereby producing stem-like T cells.

30. The method of claim 29, wherein the stem-like T cells comprise stem-like CD8+ T cells.

31. The method of claim 29-30, wherein the chemically modified double stranded molecule comprises a sequence selected from SEQ ID NOs: 1-12.

32. The method of any one of claims 29 to 31, wherein the chemically modified double stranded nucleic acid molecule is an INTASYL™.

33. The method of any one of claims 29 to 32, wherein the chemically modified double stranded nucleic acid molecule comprises at least one 2′-O-methyl modification and/or at least one 2′-O-Fluoro modification, and at least one phosphorothioate modification.

34. The method of any one of claims 29 to 33, wherein the chemically modified double stranded nucleic acid molecule induces at least 40% inhibition of PD-1 in the T cells.

35. The method of any one of claims 29 to 34, wherein the chemically modified double stranded nucleic acid molecule comprises a sense strand having the sequence set forth in SEQ ID NO: 1 and/or an antisense strand having the sequence set forth in SEQ ID NO: 2 (PD1-1).

36. The method of any one of claims 29 to 34, wherein the chemically modified double stranded nucleic acid molecule comprises a sense strand having the sequence set forth in SEQ ID NO: 1 and/or an antisense strand having the sequence set forth in SEQ ID NO: 3 (PD1-2).

37. The method of any one of claims 29 to 34, wherein the chemically modified double stranded nucleic acid molecule comprises a sense strand having the sequence set forth in SEQ ID NO: 1 and/or an antisense strand having the sequence set forth in SEQ ID NO: 4 (PD1-3).

38. The method of any one of claims 29 to 34, wherein the chemically modified double stranded nucleic acid molecule comprises a sense strand having the sequence set forth in SEQ ID NO: 1 and/or an antisense strand having the sequence set forth in SEQ ID NO: 5 (PD1-4).

39. The method of any one of claims 29 to 34, wherein the chemically modified double stranded nucleic acid molecule comprises a sense strand having the sequence set forth in SEQ ID NO: 1 and/or an antisense strand having the sequence set forth in SEQ ID NO: 6 (PD1-5).

40. The method of any one of claims 29 to 34, wherein the chemically modified double stranded nucleic acid molecule comprises a sense strand having the sequence set forth in SEQ ID NO: 1 and/or an antisense strand having the sequence set forth in SEQ ID NO: 7 (PD1-6).

41. The method of any one of claims 29 to 34, wherein the chemically modified double stranded nucleic acid molecule comprises a sense strand having the sequence set forth in SEQ ID NO: 1 and/or an antisense strand having the sequence set forth in SEQ ID NO: 8 (PD1-7).

42. The method of any one of claims 29 to 34, wherein the chemically modified double stranded nucleic acid molecule comprises a sense strand having the sequence set forth in SEQ ID NO: 9 and/or an antisense strand having the sequence set forth in SEQ ID NO: 10 (PD1-8).

43. The method of any one of claims 29 to 34, wherein the chemically modified double stranded nucleic acid molecule comprises a sense strand having the sequence set forth in SEQ ID NO: 11 and/or an antisense strand having the sequence set forth in SEQ ID NO: 10 (PD1-9).

44. The method of any one of claims 29 to 34, wherein the chemically modified double stranded nucleic acid molecule comprises a sense strand having the sequence set forth in SEQ ID NO: 12 and/or an antisense strand having the sequence set forth in SEQ ID NO: 10 (PD1-10).

45. The method of any one of claims 29 to 34, wherein the chemically modified double stranded nucleic acid molecule comprises a sense strand having the sequence set forth in SEQ ID NO: 1 and/or an antisense strand having the sequence set forth in SEQ ID NO: 10 (PD1-11).

46. The method of any one of claims 27 to 42, wherein the T cell expresses T cell factor 1 (TCF-1).

47. The method of claim 43, wherein the T cell further expresses CD95, CD45RA, CCR7, CD27, CD127, CD28, T-bet, KI-67, TOX, or any combination thereof.

48. A method comprising introducing into a T cell ex vivo a chemically modified double stranded nucleic acid molecule that is directed against a programmed cell death 1 (PD-1) gene, wherein the chemically modified double stranded nucleic acid molecule comprises at least 12 contiguous nucleotides of a sequence selected from SEQ ID NOs: 1-12.

49. A method for treating a subject suffering from a proliferative disease, the method comprising administering to the subject the immunogenic composition of any one of claims 1 to 28.

50. The method of claim 49, wherein the proliferative disease is cancer.
